SEQUENCE LISTING (15 sequences)

SEQ ID NO: 1 - protein, 12 aa, Artificial Sequence
  a fragment of CDP138
ASDLTDAFVEVK

SEQ ID NO: 2 - protein, 23 aa, Artificial Sequence
  fragment of AA 189-211 of CDP138
NEARQRLISLMSGELQRKIGLKV

SEQ ID NO: 3 - protein, 94 aa, homo sapiens
  AA 3-97 of human CDP138-C2
KLKVKIVAGRHLPVMDRASDLTDAFVEVKFGNTTFKTDVYLKSLNPQWNSEWFKFEVDDEDLQDEPLQITVLDHDTYSANDAIGKVYIDIDPLL

SEQ ID NO: 4 - protein, 95 aa, homo sapiens
  AA 157-251 of human Sytg1-C2A
QLLVGIIQAAELPALDMGGTSDPYVKVFLLPDKKKKFETKVHRKTLNPVFNEQFTFKVPYSELGGKTLVMAVYDFDRFSKHDIIGEFKVPMNTVD

SEQ ID NO: 5 - protein, 97 aa, homo sapiens
  AA 288-384 of human Sytg1-C2B
KLTVVILEAKNLKKMDVGGLSDPYVKIHLMQNGKRLKKKKTTIKKNTLNPYYNESFSFEVPFEQIQKVQVVVTVLDYDKIGKNDAIGKVFVGYNSTG

SEQ ID NO: 6 - protein, 20 aa, Artificial Sequence
  peptide of kiaa0528 used to generate rabbit pAb antibody
TKPHVEKSLQRASTDNEELC

SEQ ID NO: 7 - RNA, 19 nt, Artificial Sequence
  one of smart pool siRNAs targeting sequences for mouse 5730419I09RIK
gcaagguuaugucgauuaa

SEQ ID NO: 8 - RNA, 19 nt, Artificial Sequence
  one of smart pool siRNAs targeting sequences for mouse 5730419I09RIK
ggccacaggagucuacuua

SEQ ID NO: 9 - RNA, 19 nt, Artificial Sequence
  one of smart pool siRNAs targeting sequences for mouse 5730419I09RIK
guaagaguggucagacuaa

SEQ ID NO: 10 - RNA, 19 nt, Artificial Sequence
  one of smart pool siRNAs targeting sequences for mouse 5730419I09RIK
auacagaaauuaugccugg

SEQ ID NO: 11 - RNA, 19 nt, Artificial Sequence
  one of smart pool siRNAs targeting sequences for mouse 5730419I09RIK
gagaggaccuuccauguag

SEQ ID NO: 12 - RNA, 19 nt, Artificial Sequence
  one of smart pool siRNAs targeting sequences for mouse 5730419I09RIK
ugccauucuacaaccagga

SEQ ID NO: 13 - DNA, 21 bp, Artificial Sequence
  scrambled siRNAs duplexes
cagucgcguuugcgacuggtt

SEQ ID NO: 14 - DNA, 21 bp, Artificial Sequence
  scrambled siRNAs duplexes
ttgucagcgcaaacgcugacc

SEQ ID NO: 15 - protein, 6 aa, Artificial Sequence
  PAS motif
RXRXXX

(19) United States
(12) Patent Application Publication
(10) Pub. No.: US 2012/0204273 A1
(43) Pub. Date: Aug. 9, 2012

(54) COMPOSITIONS RELATING TO A PHOSPHOPROTEIN AND METHODS OF USE

(75) Inventor: Zhen Yue Jiang, Orlando, FL (US)

(73) Assignee: SANFORD-BURNHAM MEDICAL RESEARCH INSTITUTE, La Jolla, CA (US)

(21) Appl. No.: 13/335,760

(22) Filed: Dec. 22, 2011

Related U.S. Application Data
(60) Provisional application No. 61/426,464, filed on Dec. 22, 2010.
(60) Provisional application No. 61/431,713, filed on Jan. 11, 2011.

(51) Int. Cl. A61K 38/02; C12Q 1/48; C12N 15/85; A61K 49/00; A61P 3/10; A61P 25/00; C12N 5/09; A01K 67/027; G01N 33/53; A61K 48/00

(52) U.S. Cl. 800/3; 435/7.2; 435/15; 435/455; 514/6.7; 514/44 R; 514/7.3; 514/6.9; 514/17.7; 435/375; 800/18

(57) ABSTRACT

The invention provides methods and compositions for identifying agents that modulates 138-kDa C2 domain-containing phosphoprotein (CDP138) activity or phosphorylation levels both in vivo and in vitro. Also provided are methods and compositions to prolong the survival of neuronal cells, to ameliorate or prevent a condition associated with release of insulin from insulin producing cells and insulin-stimulated glucose metabolism, to inhibiting proliferation of a cancer cell and to inducing cell cycle arrest of a cancer cell.

This application claims the benefit of priority of U.S. Provisional application Ser. No. 61/426,464, filed Dec. 22, 2010, and U.S. Provisional application Ser. No. 61/431,713, filed Jan. 11, 2011, the entire contents of which are incorporated herein by reference.

### BACKGROUND OF THE INVENTION

The present invention relates generally to phosphoprotein compositions and methods of use, and more specifically to methods of identifying agents that modulate the phosphorylation state or activity of a phosphoprotein. In addition, the present invention relates to methods of ameliorating or preventing a condition associated with release of insulin and glucose metabolism by administering an agent that modulates a phosphoprotein. The present invention also relates to methods of inhibiting cell proliferation or inducing cell cycle arrest by modulating the activity or expression of a phosphoprotein.

Insulin regulates glucose transport into skeletal muscle and adipose tissue by increasing the cell surface localization of the glucose transporter GLUT4 (Bryant et al., 2002; Huang and Czech, 2007). In the basal state, GLUT4 is retained within specific intracellular compartments and insulin rapidly increases the movement of GLUT4 from its intracellular compartment to the plasma membrane (PM), where it captures the extracellular glucose for internalization. This effect is essential to maintain glucose homeostasis in humans, and impaired insulin action contributes to the development of type II diabetes (Saltiel and Kahn, 2001).

Insulin binding to its tyrosine kinase receptor results in tyrosine phosphorylation of insulin receptor substrate (IRS) proteins (Kasuga et al., 1982; Sun et al., 1991). Phosphorylated IRS proteins bind to and activate phosphoinositide 3-kinase (PI3K), which phosphorylates polyphosphoinositides to form PI(3,4)P2, and PI(3,4,5)P3 (Cantley, 2002). The latter recruits the protein kinase B (Akt) and the phosphoinositide-dependent kinase (PDK-1) to the PM, where PDK-1 (Alessi et al., 1997) and the mTORC2 complex phosphorylate and activate Akt (Sarbassov et al., 2005). Several lines of evidence strongly suggest that activation of the PI3K-Akt2 pathway is necessary for insulin to induce GLUT4 translocation, and for glucose transport (Martin et al., 1996; Okada et al., 1994). First, PI(3,4,5)P3 formation is required for GLUT4 insertion into the PM (Tengholm and Meyer, 2002). Second, expression of constitutively active Akt in adipocytes increases glucose uptake (Kohn et al., 1996), and conversely, a dominant negative form of Akt inhibits insulin-induced GLUT4 translocation (Hill et al., 1999; Wang et al., 1999). Third, siRNA-mediated knockdown of Akt, particularly Akt2, significantly reduces insulin-stimulated glucose transport and GLUT4 translocation in cultured cells (Jiang et al., 2003). Fourth, a diabetes-like phenotype is observed in Akt2 knockout mice (Cho et al., 2001) and fifth, an inactivating mutation in Akt2 in humans leads to the development of severe insulin resistance and diabetes mellitus (George et al., 2004).

The itinerary for GLUT4 exocytic pathway includes glucose storage vesicle (GSV) sorting, trafficking, docking, tethering, and finally fusion with the PM (Thurmond and Pessin, 2001). Accumulated evidence suggests that activation of Akt2 is involved in regulating both GSV mobilization to the periphery and membrane fusion between GSV and the PM, and this may occur through phosphorylation of different substrates. Akt2 has been shown to regulate GSV trafficking to and docking at the PM (Gonzalez and McGraw, 2006). This is consistent with the observation that Akt2 is recruited to and phosphorylates GSV components (Calera et al., 1998; Kupriyanova and Kandror, 1999). There is compelling evidence suggesting that Akt2 is also required for GSV-PM fusion (Chen et al., 2003; Koumanov et al., 2005; Ng et al., 2008; van Dam et al., 2005). Previous studies showed that Akt phosphorylation of AS160 stimulates GLUT4 trafficking (Eguez et al., 2005; Sano et al., 2003), but not the GSV-PM fusion step in cultured adipocytes (Bai et al., 2007; Jiang et al., 2008), suggesting that other Akt2 substrate(s) might be required for the last step of GLUT4 translocation.

Thus, there exists a need to identify and characterize the cellular mechanisms associated with glucose storage vesicle sorting, trafficking, docking, tethering and fusion to the PM as this would be a unique target for the development of new drug candidate for the treatment of diseases associated with glucose metabolism and other related conditions. As disclosed herein, the invention provides such a unique target and related methods of use.

### SUMMARY OF INVENTION

In some embodiments, the invention provides methods for identifying an agent that modulates 138-kDa C2 domain-containing phosphoprotein (CDP138) activity by (a) contacting a cell with a candidate agent, wherein the cell expresses a CDP138 polypeptide or active fragment thereof and (b) detecting CDP138 activity, wherein increased or decreased CDP138 activity in said cell compared to a control cell indicates that said candidate agent is an agent that modules CDP138 activity. In some aspects, contacting the cell occurs in vitro or in vivo. In some aspects, the CDP138 activity is binding Ca²⁺, binding lipid membranes or inducing fusion of GLUT4 vesicles with a plasma membrane. In some aspects, the CDP138 activity is increased, or alternatively the activity is decreased.

In some embodiments, the invention provides methods for identifying an agent that alters phosphorylation of CDP138 by (a) contacting CDP138 or a fragment thereof with a candidate agent under conditions that allow phosphorylation of the CDP138 or fragment thereof and (b) detecting the phosphorylation level of the CDP138 or fragment thereof, wherein altered phosphorylation levels of the CDP138 or fragment thereof indicates that the candidate agent effectively alters the phosphorylation of CDP138. In some aspects contacting the CDP138 occurs in vitro. In some aspects, the altered phosphorylation of CDP138 is increased phosphorylation, or alternatively decreased phosphorylation.

In some embodiments, the invention provides methods to prolong survival of a neuronal cell by introducing into the neuronal cell a nucleic acid molecule encoding CDP138 or an active fragment thereof, whereby expression of the CDP138 or an active fragment thereof prolongs the survival of the cell. In some aspects of the invention, survival of the cell occurs in vitro or in vivo. In some aspects, the neuronal cell is a human neuronal cell. In some aspects of the invention, the neuronal cell is a neuronal precursor cell or neuronal stem cell.

In some embodiments, the invention provides a method for ameliorating or preventing a condition associated with release of insulin from insulin producing cells and insulin-stimulated glucose metabolism in an individual by administering an effective amount of an agent that modulates 138-kDa C2 domain-containing phosphoprotein (CDP138) activity in the individual afflicted with the condition, whereby the condition is ameliorated or prevented. In some aspects, the agent is a chemical compound, such as a small molecule, a nucleic acid or a protein. In some aspects, the condition is diabetes mellitus type 1, diabetes mellitus type 2 and a neurodegenerative disease, such as Alzheimer's disease, Parkinson's disease, epilepsy, dementia, schizophrenia, depression, anxiety or autism spectrum disorder.

In some embodiments, the invention provides a method for inhibiting proliferation of a cancer cell by contacting the cancer cell with an agent that modulates 138-kDa C2 domain-containing phosphoprotein (CDP138) activity in the cancer cell, whereby increased activity of the CDP138 inhibits the cancer cell from dividing, thereby inhibiting proliferation of said cancer cell. In some aspects contacting the cell occurs in vitro or in vivo. In some aspects, the agent is a chemical compound, such as a small molecule, a nucleic acid or a protein.

In some embodiments, the invention provides a method for inducing cell cycle arrest of a cancer cell by introducing into the cancer cell a nucleic acid molecule encoding 138-kDa C2 domain-containing phosphoprotein (CDP138) or an active fragment thereof, whereby expression of the CDP138 or active fragment thereof induces cell cycle arrest of the cancer cell. In some aspects, contacting the cell occurs in vitro or in vivo.

### BRIEF DESCRIPTION OF THE DRAWINGS

FIG. 1, panels A-D, show an uncharacterized C2 domain-containing protein encoded by 5730419I09Rik is a novel phosphoprotein identified in insulin-stimulated adipocytes using a SILAC phosphoproteomic approach. (A): Schematic procedure for SILAC quantitative proteomics used for identification and quantification of peptide from CDP138 (5730419I09Rik). Top right panel: quantification of CDP138 peptide from different groups of adipocytes. Lower right panel: Schematic diagram of CDP138 and the identified phosphorylation sites. (B): Confirmation of CDP138 phosphorylation induced by insulin. CHO-T cells expressing HA-CDP138, or differentiated 3T3-L1 adipocytes were treated with or without insulin (100 nM) for 15 min. A third sample was pretreated with wortmannin (100 nM, WM) for 20 min or LY294002 (50 μM, LY) for 1 hour before insulin stimulation. Left panel: HA-CDP138 was immunoprecipitated with anti-HA Ab from CHO-T cells and blotted first with the PAS phospho-Akt substrate motif Ab, and then reblotted with an anti-HA Ab. Right panel: An anti-CDP138 peptide Ab was used for immunoprecipitation of CDP138 from cell lysates of adipocytes followed by immunoblotting with the same Ab. CDP138, pS473-Akt and Akt protein were also detected with total cell lysates. The arrows indicate the insulin-stimulated CDP138 mobility shift. (C): Constitutively active Akt2 (myr-HA-Akt2) directly phosphorylates HA-CDP138 in vitro kinase assays (left panel) as described in Experimental Procedures. Mass spectrometry identification of Ser197 residue in CDP138 as the phosphorylation target of myr-HA-Akt2 (right panel) by Higher Energy Collision Dissociation (HCD-MS/MS). (D): CDP138 protein expression in insulin sensitive tissues from C57B/6J male lean and ob/ob mice. Whole cell extracts (25 μg total protein) from different tissues (lean mice, left panel) and epididymal fat pads from both ob/ob and lean mice (24 weeks old, Jackson Lab, Bar Harbor, Me.; middle panel) were analyzed by immunoblotting with antibodies against CDP138, IRS-1, Akt, Erk1/2, and β-actin. Right panel: quantification of CDP138 protein levels in fat tissues from lean and obese mice. Data are mean±SEM, **P<0.01 lean vs ob/ob mice (n=4). WAT, white adipose tissue; BAT, brown adipose tissue.

FIG. 2, panels A-C show knockdown of CDP138 in 3T3-L1 adipocytes inhibits insulin-stimulated glucose transport (A) and myc-GLUT4-GFP translocation (B), but not endogenous GLUT4 movement to the periphery detected in TIRF zone (C). (A): Differentiated adipocytes at day 5 were transfected with siRNAs against mouse 5730419I09Rik or the scrambled siRNA (Scr) as described earlier (Jiang et al., 2003) for 60 hrs, then serum starved overnight. Cells were then treated with or without insulin (1 nM and 100 nM) for 30 min for the glucose uptake assay, or 15 min for immunoblotting of CDP138, pAkt, Akt and β-actin. Glucose transport data are presented as mean±SD of 4 independent experiments. *P<0.05 vs Scr Insulin (1 nM) group; **P<0.01 vs Scr Insulin (100 nM) group. (B): Day 5 adipocytes were transfected with siRNAs and myc-GLUT4-GFP for 60 hrs and then serum starved overnight. Cells were then treated with insulin (1 nM) for 20 min. Cell surface Myc-GLUT4-GFP was detected with anti-myc monoclonal Ab (9E10) and Alexa Fluor 568-labeled goat anti-mouse IgG in non-permeablized cells. The Myc signal and GFP signal were quantified as previously described (Jiang et al., 2002). Data presented are representative microscopic images and mean±SD of about 160 GFP-positive cells in each group from three independent experiments. **P<0.01 vs Scr Insulin (1 nM) group. (C): Endogenous GLUT4 accumulation in the TIRF zone in fixed adipocytes treated with or without insulin for 20 min. GLUT4 was detected with a goat anti-GLUT4 Ab and Alexa Fluor 488-conjugated donkey anti-goat Ab in permeabilized cells. Top and middle panels show 100 nm TIRF zone and representative GLUT4 TIRFM images, respectively. Data are mean±SD of 3 independent experiments. **P<0.01 vs Scr Insulin groups.

FIG. 3, panels A-C, show knockdown of CDP138 in live 3T3-L1 adipocytes inhibits insulin-stimulated membrane fusion between GLUT4 storage vesicles (GSV) and the PM, but not GLUT4-EGFP trafficking to the TIRF zone. (A): Schematic illustration of the molecular probes and TIRF microcopy-based live cell assays for GLUT4 trafficking and GSV-PM fusion. (B) and (C): The effect of CDP138 knockdown on insulin-stimulated IRAP-pHluorin insertion into the PM (B), and accumulation of GLUT4-EGFP in the TIRF zone (C). Adipocytes (day 4) were transfected by electroporation with plasmid DNA encoding IRAP-pHluorin or GLUT4-EGFP, together with either the scrambled siRNA or smartPool siRNA against mouse 5730419I09Rik. Cells were reseeded on glass-bottomed dishes for 72 hrs, serum starved for 2 hr then stimulated with 100 nM insulin for 30 min. Analyses were performed in a cell warmer adapted for a Nikon TiE with fully motorized combined dual laser (488 and 561 nm). Images were acquired every 3 min immediately after addition of insulin and analyzed as described in the Supplementary Experimental Procedures. Perfect focus system and multiple points capture program (NIS Element) were used to acquire images from multiple positively transfected cells at each time point. Data are mean±SEM of 159 cells (Scrambled siRNA) or 154 cells (CDP138 siRNA) in the GLUT4-EGFP trafficking assay; and 125 cells (Scrambled siRNA) or 120 cells (CDP138 siRNA) in the GSV-PM fusion assay. **P<0.01 CDP138 siRNAs vs scrambled siRNA at all time points except 3 min after addition of insulin.

FIG. 4, panels A-D, show the purified C2 domain from CDP138 is capable of binding Ca²⁺ ions and lipid membranes. (A): Diagram and amino acid alignment of the C2 domains of CDP138 (CDP138-C2) and synaptotagmin-1 (Sytg1-C2A & Sytg-C2B). β: beta strands; α: alpha helix; loop: coil loop. The conserved potential Ca²⁺-binding aspartate residues are highlighted. (B): Gel images of purified MBP (42 kDa), MBP-C2-WT domain, and MBP-C2-5DA mutant. (C): Calcium binds to the wild type C2-domain but not to the 5DA or the MBP proteins. Change in tryptophan fluorescence intensity at 340 nm as a signal of calcium interaction with the MBP-C2-WT fusion protein, MBP-C2-5DA mutant fusion protein, and MBP measured at 37° C. The calcium-binding isotherm was constructed as described herein. Calcium exerts a biphasic effect on tryptophan fluorescence of the wild type protein, but has little effect on the 5DA or MBP proteins. The curve of calcium binding to MBP-C2-WT was constructed using two independent binding sites per wild type C2 domain, with dissociation constants of K_{D,1}=0.03 μM and K_{D,2}=15 μM. (D): Fluorescence resonance energy transfer (RET) from protein tryptophan residues to Py-PE in membrane indicates membrane binding by the MBP-C2 WT fusion protein but not by the MBP-5DA-C2 mutant or MBP proteins. Change in tryptophan fluorescence intensity as a function of lipid concentration, corrected for the effect of membranes without energy acceptor Py-PE, measured at 37° C. The solid line describing membrane binding of MBP-C2-WT was simulated using a lipid-to-protein stoichiometry N=20 and a dissociation constant K_{D}=0.06 mM.

FIG. 5, panels A-C, show CDP138 co-localizes with phospho-Akt (A & B), and is required for constitutively active myr-Akt2-induced GLUT4 translocation (C). A & B: HA-CDP138-WT was transfected into adipocytes (A) and CHO-T cells (B) for 48 hrs before serum starvation overnight. Cells were then treated with or without insulin (100 nM) for 10 min. Cells were fixed and permeabilized before immunostaining with mouse anti-HA and rabbit anti-phospho-Akt (S473) antibodies followed by goat anti-mouse (Alexa Fluor568) and goat anti-rabbit (Alexa Fluor488) secondary antibodies, respectively. The white arrow indicates co-localization of phospho-Akt and HA-CDP138. C: Differentiated adipocytes were transfected by electroporation with the scrambled siRNA or CDP138 siRNAs together with plasmid DNAs encoding myc-GLUT4-GFP and myr-HA-Akt2 or HA-empty vector. Cells were reseeded for 60 hours before serum starvation overnight. Myc-GLUT4 translocation assays were carried out with TIRF microscopy as described in the Experimental Procedures. Data are mean±SEM of four independent experiments. **P<0.01 myr-Akt2/CDP138 siRNA vs myr-Akt2/Scr siRNA. Scale bar=5 μm.

FIG. 6, panels A-D, show the effects of CDP138-ΔC2, CDP138-5DA and CDP138-S197A mutants on myc-GLUT4-GFP translocation and GSV-PM fusion in 3T3-L1 adipocytes. Plasmid DNAs for pCMV5-HA, HA-CDP138-WT, HA-CDP138-ΔC2, HA-CDP138-5DA, or HA-CDP138-S197A were transfected by electroporation into adipocytes together with the myc-GLUT4-GFP expression vector. Cells were reseeded for 48 hrs and serum-starved overnight. Cells were then treated with or without insulin (100 nM) for 30 min, before immunostaining with rabbit anti-myc and mouse anti-HA antibodies. (A): Schematic diagram for CDP 138 (wild type and mutants) and representative images of the myc-GLUT4-GFP translocation assay using TIRF microscopy. (B): GLUT4 translocation to the cell surface of fixed adipocytes is shown as the ratio of surface TIRF myc signal to total Epi GFP. Data are presented as mean±SEM of three independent experiments. **P<0.01 ΔC2, 5DA, or S197A vs HA vector (C) & (D): The effects of CDP138-mCherry constructs on insulin-induced GSV-PM fusion and GLUT4-EGFP trafficking in live adipocytes, respectively. Data are expressed in arbitrary units as the ratio of pHluorin or EGFP intensity to the basal intensity at time zero. Data are mean±SEM of three independent experiments. *P<0.05 S197A vs mCherry vector at 6 min incubation with insulin; **P<0.01 5DA or S197A vs mCherry vector at all time points except 3 and 6 min after addition of insulin.

FIG. 7, panels A-D, show CDP138 is co-localized with GLUT4 at the PM (A) and dynamically associated with the PM fractions (B) and GLUT4 vesicles (C). HA-CDP138-WT plasmid DNA was transfected alone into a CHO-T cell line stably expressing myc-GLUT4-GFP, or into adipocytes together with the myc-GLUT4-GFP vector. Forty-eight hr later, serum starved cells were treated with or without insulin for 10 min before immunofluorescent staining with anti-HA Ab, as described for FIG. 5. (B): Subcellular membrane fractionation with iodixanol gradients was performed as described in Experimental Procedures, followed by immunoblotting with antibodies against CDP138, p-Akt and GLUT4. (C): GLUT4 vesicles were enriched by immunoabsorption with anti-GLUT4 Ab (1F8) from adipocytes after removal of nuclear fraction as described herein. Samples were then immunoblotted with anti-CDP138 and anti-GLUT4 antibodies. Images are representative of three independent experiments (A, B & C). (D): CDP138 phosphorylation and interaction with the PM and GSV is a potential cellular mechanism that links Akt2 activation to the process of GSV-PM fusion. Scale bar=5 μm

FIG. 8, panels A-B, show Akt1/2 Inhibitor (Akti1/2) blocks insulin-stimulated GSV-PM fusion in live adipocytes and myc-GLUT4-GFP translocation to cell surface in fixed cells. (A): In live cell experiment, differentiated adipocytes were transfected with IRAP-pHlorin by electroporation and then seeded on glass-bottomed dishes for 24 hrs before serum starvation for 2 hr and insulin stimulation (100 nM) for 30 min. Analyses were performed in a cell warmer adapted to the Nikon TiE with fully motorized combined dual laser (488 and 568 nm). Images were acquired every 3 min before and after addition of insulin and analyzed as described in the Supplementary Experimental Procedures. For inhibitor studies, Akti1/2 (5 μM) was added to the cell culture medium for 1 h before adding 100 nM insulin for 30 min. The ratio of pHluorin at each time point over the basal pHlorine intensity at the TIRF zone was used as the index of membrane fusion rate. Data are mean+/−SEM more that 80 cells. (B): For fixed cell study, myc-GLUT4-GFP plasmid DNA was transfected by electroporation into adipocytes. Cells were reseeded for 48 hrs and serum-starved overnight. Cells were treated with or without Akti1/2 (50 μM) for 1 hr then treated with or without insulin (100 nM) for 30 min before immunostaining with rabbit anti-myc antibody. GLUT4 translocation to the cell surface of fixed adipocytes is shown as the ratio of surface TIRF myc signal to total Epi GFP. Data are presented as mean±SEM of three independent experiments. **P<0.01 Akti1/2 vs Insulin alone group.

FIG. 9, panels A and B, show detection of calcium and lipid membrane binding capability of the purified C2 domain. (A): Calcium binds to MBP-C2-WT, but not MBP-C2-5DA or MBP proteins. Dependence of tryptophan fluorescence spectra of MBP-C2-WT, MBP-C2-5DA, and MBP proteins on CaCl₂ concentration. Change of color from blue to red corresponds to Ca²⁺ concentrations from 0 to 240 μM. The excitation wavelength was 290 nm, and spectra were measured at 37° C. Other details are described in Supplementary Experimental Procedures, and analysis of Ca²⁺ binding to MBP-C2-WT is described in FIG. 4 and in the main text. (B): Resonance energy transfer (RET) from tryptophan to pyrene-phosphatidylethanolamine (Py-PE) indicates membrane binding of the MBP-C2-WT protein but not the MBP-C2-5DA or MBP proteins. Changes in tryptophan fluorescence spectra of MBP-C2-WT, MBP-C2-5DA, and MBP proteins upon titration with phospholipid vesicles without (upper row) and with 2% Py-PE (lower row). Excitation was at 290 nm. Change of color from blue to red corresponds to total lipid concentration from 0 to 500 μM, and protein concentration was 1.10 to 1.65 μM. Lipid composition of the membranes and other details are described in Supplementary Experimental Procedures, and analysis of membrane binding to MBP-C2-WT is described in FIG. 4 and in the main text.

FIG. 10, shows, overexpressed CDP138 mutant lacking Ser197, but not Ser200, phosphorylation site blocks insulin-stimulated myc-GLUT4-GFP translocation to the surface of adipocytes. Myc-GLUT4-GFP plasmid DNA was transfected by electroporation into adipocytes together with pcMV5-HA vector, HA-CDP138-S197A or HA-CDP138-S200A vectors. Cells were reseeded for 48 hrs and serum-starved overnight. Cells were then treated with or without insulin (100 nM) for 30 min, before immunostaining with rabbit anti-myc antibody. GLUT4 translocation to the cell surface of fixed adipocytes is shown as the ratio of surface TIRF myc signal to total Epi GFP. Data are presented as mean±SEM of three independent experiments. **P<0.01 S197A insulin vs pCMV5 insulin group.

FIG. 11 shows the survival of differentiated PC12 neuronal cells upon transfection with wild-type CDP138 (WT), dominant negative mutant CDP138-5DA (5DA) or empty vector (EMPTY) followed by contacting with neuronal growth factor (NGF). Cells were serum starved for 6 hours (6 H), 14 hours (14 H) or 24 hours (24 H) before counting the number of cells in two wells.

FIG. 12 shows the number of big surviving differentiated PC12 neuronal cells upon transfection with wild-type CDP138 (WT), dominant negative mutant CDP138-5DA (5DA) or empty vector (CTRL) followed by contacting with neuronal growth factor (NGF). Cells were serum starved for 24 hours before counting the number of bid surviving differentiated neuronal cells in two wells.

FIG. 13, panels A and B, show the expression pattern of CDP138 in various tissues and the effects of overexpressed wild-type and mutant CDP138 in neuronal growth factor (NGF)-induced neurite outgrowth in PC12 cells. A: CDP138 is highly expressed in the brain and distributed in the neurite and synapse of neuronal cells. 30 μg of total protein was assayed for all tissues, except only 15 μg of total protein from brain tissue was assayed in the immunoblotting of panel A. Exemplary protein distribution of CDP138-mCherry in a PC12 cell is also shown. B: Overexpressed CDP138-WT enhances neurite growth, whereas the CDP138-5DA mutant had the opposite effect. The y axis shows the fold over cell body length resulting from NGF-induced neurite outgrowth.

FIG. 14, shows overexpressed CDP138 inhibits cell division in CHO-T cells (proliferative cells) by formation of large cell bodies with multiple nuclei. Column 1 shows green fluorescence from pAk transfected CHO-T cells. Column 2 shows red fluorescence from HA-CDP138 transfected cells. Column 3 shows a merged image of co-transfected CHO-T cells from column 1 and 2. Large cells bodies with multiple nuclei were formed in HA-tagged CDP138 positive CHO-T cells.

FIG. 15, panels A-D, show a gene trap strategy for generating CDP138 (5730419I09Rik) knockout mice and the genotyping mutant mice. (A) ES cell clones were created by TIGM using genetrap strategy. Three clones ES cell lines with insert trap between 1st and 2nd Exons of 5730419I09Rik gene were selected for injection blastocysts of C57BL6N. Chimeras were identified by coat color and the agouti mice were backcrossed into C57BL6N in order to test for germline transmission. F1 heterozygotes were bred in order to propagate the line. (B) Extensive genotyping was performed by PCR in order to confirm the presence of the insertion. WT mice produce 182 bp PCR product and homozygous mutant mice have 150 bp product. B: (lower panel), tissue lysates (50 ug protein) from CDP138−/− and WT littermate were used for immunoblotting with anti-CDP138 antibody. (C) Fasting blood glucose levels in WT and CDP138−/− mice after challenging with high fat diet for 4 weeks. Data is mean+/−SEM (WT n=4, CDP138−/− n=4). **P<0.01. (D) Monitoring body weight of both wild type (WT) and CDP138 knockout (KO) mice fed with normal chow diet and 60% high-fat diet.

FIG. 16, upper and lower panels, show CDP138 knockout mice are prone to high-fat diet-induced myocardial hypertrophy and fibrosis. Upper panel: CDP138−/− (KO) and wild type (WT) littermate were fed with normal chow diet or 60% HFD for 12 weeks before hearts were harvested for imaging. Scale bar=1 mm. Lower panel: Masson's trichrome staining of myocardial section from mid-left ventricular wall of female WT and KO and WT HFD mice. Red: muscle fibers; blue: collagen; light red: cytoplasm, and dark brown: cell nuclei. Arrows indicate fibrosis. Scale bar=50 μm.

### DETAILED DESCRIPTION OF THE INVENTION

The invention described herein is based, in part, on the identification and characterization of the previously uncharacterized KIAA0528 protein encoded by the kiaa0528 gene as a phosphoprotein in cells treated with insulin, referred to herein as 138-kDa C2 domain-containing phosphoprotein (CDP138). Additionally, siRNA-induced gene specific knockdown of CDP138 leads to the inhibition of insulin-stimulated glucose transporter GLUT4 translocation and glucose transport in cultured adipocytes, as described herein. Further biochemical and cell biology studies demonstrated that CDP138 is phosphroylated by Akt2 and CaMKII, both activated in the cells treated with insulin and required for insulin-stimulated glucose metabolism, as described herein. Together, this data shows that the protein CDP138 is regulated by insulin and is crucial for glucose metabolism under physiological conditions. CDP138 protein level is significantly reduced in tissues of obese diabetic animal models, suggesting the physiological and pathological importance of the novel gene.

The invention described herein is also based, in part, on the finding that overexpression of CDP138, particularly its mutant lacking the phosphorylation sites, was shown in proliferative cell line to block cell division by the formation of multiple nucleus cells, as described herein. Thus, this protein is involved in the regulation of the cell cycle and can be a drug target for cancer given that CDP138 is likely a CDK substrate and potentially interacts with several tumor suppressive factors.

The invention described herein is also based, in part, on the finding that overexpression of wild type CDP138 protein was shown to help the neuronal cell (PC12) survive longer under starvation conditions, whereas overexpression of a mutant form of this novel protein resulted in cell death. These results show that CDP138 can have a protective effect to neuronal cells.

The invention described herein is still further based, in part, on the finding that the functions of CDP138 show that it can be a drug target for screening lead chemical compounds in the treatment of diabetes and cancer, as described herein. For example, searching for small molecules that selectively enhance CDP138 function and expression can be a useful strategy for the treatment of diabetes, cancer and neurodegenerative diseases. Accordingly, the invention provides that CDP138 is a drug target for diabetes and cancer and provides an approach for searching for new drugs for the treatment of diabetes, cancer and neurodegenerative diseases.

The “138-kDa C2 domain-containing phosphoprotein” or “CDP138” refers to the previously uncharacterized KIAA0528 polypeptide encoded by the kiaa0528 gene, which is also known in the art as the hypothetical protein LOC9847 and DKFZp779N2044. KIAA0528 is a protein that was first identified in the in silico analysis of long cDNAs isolated in the Kazusa cDNA sequencing project. Prior to the experiments presented herein, the function of KIAA0528 had not been characterized. By similarity, KIAA0528 contains a C2 domain, a domain that is found in proteins that bind phospholipids. The C2 domain is also present in a family of proteins involved in synaptic vesicle trafficking. A CDP138 polypeptide or protein that can be used in the methods disclosed herein include those encoded by the kiaa0528 gene from a variety of organisms, such as, but not limited to, human, mouse, rat, chicken, dog, chimpanzee or any homologue or ortholog thereof. Exemplary nucleotide sequences encoding the CDP138 protein and their corresponding amino acid sequences can be found in the GenBank database maintained by the Nation Center for Biotechnology Information by the following gene accession and gene identification numbers: nucleic acid sequences—BC117143 (GI: 109658767) or NM_{—}014802.1 (GI: 29789059) from the organism Homo sapiens, XP_{—}866112.1 (GI: 73997028) from the organism Canis familiaris; XP_{—}001147809.1 (GI: 114645641) from the organism Pan troglodytes; XM_{—}342783.3 (GI: 109472758) from the organism Rattus norvegicus; NM_{—}029081.1 (GI: 30794171) from the organism Mus musculus; and XM_{—}416427.2 (GI: 118083099) from the organism Gallus gallus; amino acid sequences—AAI17144.1 (GI: 109658768) or NP_{—}055617.1 (GI: 29789060) from the organism Homo sapien, XP_{—}866112.1 (GI: 73997028) from the organism Canis familiaris; XP_{—}001147809.1 (GI: 114645641) from the organism Pan troglodytes; XP_{—}342784.2 (GI: 62648316) from the organism Rattus norvegicus; NP_{—}083357.1 (GI: 30794172) from the organism Mus musculus; and XP_{—}416427.2 (GI: 118083100) from the organism Gallus gallus, which are all herein incorporated by reference. The invention also provides that a CDP138 polypeptide useful in the methods disclosed herein includes one of the various isoforms of KIAA0528 protein known in the art. A protein isoform is any of several different forms of the same protein. Different forms of a protein may be produced from related genes, or may arise from the same gene by alternative splicing. For example, the KIAA0528 protein from Homo sapiens has 10 spliced and one unspliced mRNAs that putatively encode functional proteins. Altogether 11 different isoforms (5 complete, 1 COOH complete, 5 partial) are produced, some of which contain the C2 calcium-dependent membrane targeting domain (NCBI—AceView: gene: KIAA0528: Homo sapiens complex locus KIAA0528, encoding KIAA0528, 2011. (http://www.ncbi.nlm.nih.gov/IEB/Research/Acembly/av.cgi?db=human&q=KIAA0528). The remaining 4 mRNA variants (1 spliced, 3 unspliced; 3 partial) appear not to encode functional proteins (NCBI—AceView: gene: KIAA0528: supra).

The term “agent” or “candidate agent” or “drug candidate” or grammatical equivalents as used herein describes any molecule, either naturally occurring or synthetic, e.g., protein, oligopeptide (e.g., from about 5 to about 25 amino acids in length, or alternatively from about 10 to 20 or 12 to 18 amino acids in length, or alternatively 12, 15, or 18 amino acids in length), small organic molecule, polynucleotide, RNAi or siRNA, asRNA, oligonucleotide, etc. An agent is a molecule that can be tested in an assay to identify the ability of the agent to modulate the activity of CDP138. The agent can be a member of a library of test compounds, such as a combinatorial or randomized library that provides a sufficient range of diversity. The agent can be optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. Often, high throughput screening methods are employed for such an analysis.

“Inhibitors,” “activators,” and “modulators” of expression or of activity are used to refer to inhibitory, activating, or modulating molecules, respectively, identified using in vitro and in vivo assays for expression or activity, e.g., ligands, agonists, antagonists, and their homologs and mimetics. The term “modulator” includes inhibitors and activators. Inhibitors include agents that bind to, partially or totally block stimulation or enzymatic activity, decrease, prevent, delay activation, inactivate, desensitize, or down regulate the activity of CDP138, for example, antagonists. Activators can be agents that bind to, stimulate, increase, activate, facilitate, enhance activation or enzymatic activity, sensitize or up regulate the activity of CDP138, for example, agonists. Modulators include naturally occurring and synthetic ligands, antagonists, agonists, small chemical molecules and the like. Assays to identify inhibitors and activators include, e.g., applying putative modulator compounds to cells, in the presence or absence of CDP138 and then determining the functional effects on CDP138 activity. Samples or assays comprising CDP138 that are treated with a potential activator, inhibitor, or modulator are compared to control samples without the inhibitor, activator, or modulator to examine the extent of effect. Control samples (untreated with modulators) are assigned a relative activity value of 100%. Inhibition is achieved when the activity value of CDP138 relative to the control is at most 80% of the activity value in the control samples, or alternatively at most 60%, or alternatively at most 40%, or alternatively at most 20, or alternatively at most 10%. Activation is achieved when the activity value of CDP138 relative to the control is 110%, optionally 150%, optionally 200-500%, or 1000-3000% higher.

As used herein, the phrase “active fragment” when used in reference to the CDP138 polypeptide refers to an amino acid sequence that is a portion or fragment of a CDP138 polypeptide as described herein, which maintains one or more of the activities of the full length or wild-type CDP138 polypeptide. Such activities are described herein. Non-limiting examples include, but are not limited to, binding to Ca²⁺, binding to lipid membranes, or inducing fusion of GLUT4 vesicles with the plasma membrane of a cell.

In some embodiments, the invention provides methods for screening and identifying an agent that modulates one or more of the CDP138 activities. Accordingly, in some embodiments, the invention provides a method for identifying an agent that modulates CDP138 activity by (a) contacting a cell with a candidate agent, wherein the cell expresses a CDP138 polypeptide or active fragment thereof and (b) detecting CDP138 activity, wherein increased or decreased CDP138 activity in said cell compared to a control cell indicates that the candidate agent is an agent that modules CDP138 activity. Methods for screening candidate agents are well known in the art. Non-limiting examples of such methods include high throughput screening methods as described in U.S. Publication 2008/0009019, in vivo screening methods as described in U.S. Pat. No. 7,211,375 or any of the methods as reviewed in Kodadek, Nature Chemical Biology, 6:162-165 (2010). Accordingly, in some aspects of the invention, contacting the cell occurs in vitro or in vivo. In some aspects, the CDP138 activity is binding Ca²⁺, binding lipid membranes or inducing fusion of GLUT4 vesicles with a plasma membrane. Any number of techniques or methods can be used for measuring CDP138 activity, which includes the methods as described in the Examples below or any equivalent thereof. In some aspects, the CDP138 activity is increased, or alternatively the activity is decreased.

In a further aspect of the invention, the cells are contacted with insulin or a functional equivalent thereof. An insulin analog is an altered form of insulin, different from any occurring in nature, but still available to the body for performing the same action as insulin in terms of glycemic control. Examples of functional equivalent to insulin include, but are not limited to, NPH insulin, Lispro insulin, Aspart insulin, Glulisine insulin, Glargine insulin or Detemir insulin. In some aspects of the invention, the candidate agent is selected from a chemical compound, such as a small molecule, a nucleic acid or a protein. In some aspects, the cell is a pancreatic cell, a heart cell, a cancerous cell, a neuronal cell, a muscle cell, a liver cell, an adipocyte, a blood cell or an embryonic stem cell.

In some aspects of the invention, the CDP138 polypeptide has the amino acid sequence of human CDP138, mouse CDP138, rat CDP138, chicken CDP138, dog CDP138 or chimpanzee CDP138. In some aspects, the CDP138 polypeptide has a mutation or a modification in the amino acid sequence of the CDP138 polypeptide, such as, but not limited to S197, S260, S262, S295, T314, T330, S597, S652, S726, S855, D19, D26, D76, D78, D84 and the C2 domain of CDP138. Exemplary mutations in the CDP138 polypeptide include the naturally occurring or experimentally induced replacement of one or more amino acids in a protein with another. If a functionally equivalent amino acid is substituted, the protein may retain wild-type activity. However, if a functionally non-equivalent amino acid is substituted, the activity associated with that amino acid or region of the protein can be inhibited or lost if the substitution occurring at a position that participates in the activity or affect the structure of a protein domain that participates in an activity. One skilled in the art readily understands the meaning of a functionally equivalent or functionally non-equivalent amino acid substitution. Experimentally induced substitution can be used to study enzyme activities and binding site properties as is well know to those skilled in the art. A modification to one or more amino acid residues includes, but is not limited to, posttranslational modification of amino acids, which can extend the range of functions of the protein by attaching to it other biochemical functional groups such as acetate, phosphate, various lipids and carbohydrates, by changing the chemical nature of an amino acid or by making structural changes, like the formation of disulfide bridges. In some aspects, the CDP138 polypeptide is recombinantly expressed in the cells.

In some embodiments, the invention provides a method for identifying an agent that alters phosphorylation of 138-kDa C2 domain-containing phosphoprotein (CDP138) by (a) contacting CDP138 or a fragment thereof with a candidate agent under conditions that allow phosphorylation of the CDP138 or fragment thereof and (b) detecting the phosphorylation level of the CDP138 or fragment thereof, wherein altered phosphorylation levels of the CDP138 or fragment thereof indicates that the candidate agent effectively alters the phosphorylation of CDP138. Methods for detecting protein phosphorylation are well known in the art and any number of which can be used in the methods of the invention. Kinase activity within a biological sample is commonly measured in vitro by incubating the kinase with an exogenous substrate in the presence of ATP. Measurement of the phosphorylated substrate can be assessed by several reporter systems including colorimetric, radioactive, or fluorometric detection. Direct detection of phosphorylated CDP138 can provide a more detailed analysis of the cellular response to an external stimulus, as identification of a phosphopeptide provides information regarding the expression and the functional state of CDP138. Non-limiting examples of such methods are described in the Examples below and also include phosphor-specific antibody detection, western blotting, enzyme-linked immunosorbent assay (ELISA), cell-based ELISA, intracellular flow cytometry, immunocytochemistry/immunohistochemistry and mass spectrometry. Thus, in some aspects of the invention, contacting the CDP138 occurs in vitro. In some aspects, the phosphorylation of CDP138 is located at Ser197. In some aspects, the altered phosphorylation of CDP138 is increased phosphorylation, or alternatively decreased phosphorylation. In some aspects, the candidate agent is a chemical compound, such as a small molecule, a nucleic acid or a protein.

In some aspects, the CDP138 is human CDP138, mouse CDP138, rat CDP138, chicken CDP138, dog CDP138 or chimpanzee CDP138. In some aspects the CDP138 has a mutation or a modification at a position in the amino acid sequence of CDP138, such as, but not limited to, S197, S260, S262, S295, T314, T330, S597, S652, S726, S855, D19, D26, D76, D78, D84 or the C2 domain of CDP138.

In a further aspect of the invention, the contacting of the CDP138 or fragment thereof includes contacting with a kinase. Non-limiting examples of such kinases that are useful to phosphorylate CDP138 in the methods described herein include Akt1, Akt2, cyclin-dependent kinase (CDK), mammalian target of rapamycin (mTOR) and Calmodulin-dependent protein kinase II (CaMKII) to phosphorylate CDP138.

In some embodiments, the invention provides a method to prolong survival of a neuronal cell by introducing into the neuronal cell a nucleic acid molecule encoding CDP138 or an active fragment thereof, whereby expression of the CDP138 or an active fragment thereof prolongs the survival of the cell. Methods for introducing a nucleic acid molecule encoding CDP138 are well known in the art and any number of such methods can be used in the invention described herein. Examples of such methods are reviewed in Karra and Dahm, The Journal of Neuroscience, 30(18):6171-6177 and include electrical transfection, chemical transfection, virus-based transfection, physical transfection and the like. A polynucleotide encoding CDP138 can be delivered to a cell or tissue using a gene delivery vehicle.

“Gene delivery,” “gene transfer,” “transducing,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein. Such methods are well known to those skilled in the art (Sambrook et al., Molecular Cloning: A Laboratory Manual, (Third Edition) (2001) and Ausubel et al. eds. (2007) Current Protocols in Molecular Biology).

In some aspects of the invention, survival of the cell occurs in vitro or in vivo. Survival of the cells can be measured using a number of methods known in the art. Non-limiting example of such methods are described in the Examples below and include in vitro counting of cells in a given sample, measuring cell apoptotic or necrotic markers as described in U.S. Pat. No. 5,750,360 or Schmid et al., J. Immunol. Methods, 170(2):145-157 (1994). In some aspects, the neuronal cell is a human neuronal cell. In some aspects of the invention, the neuronal cell is a neuronal precursor cell or neuronal stem cell.

In some aspects of the invention, the method of prolong survival of a neuronal cell by introducing into the neuronal cell a nucleic acid molecule includes a nucleic acid molecule encoding a polypeptide such as, but not limited to, a human CDP138, mouse CDP138, rat CDP138, chicken CDP138, dog CDP138 or chimpanzee CDP138, as described herein. In some aspects of the invention, expression of said CDP138 is overexpression.

In some embodiments, the invention provides a method for ameliorating or preventing a condition associated with release of insulin from insulin producing cells and insulin-stimulated glucose metabolism in an individual by administering an effective amount of an agent that modulates 138-kDa C2 domain-containing phosphoprotein (CDP138) activity in the individual afflicted with the condition, whereby the condition is ameliorated or prevented. In some aspects, the agent is a chemical compound, such as a small molecule, a nucleic acid or a protein. In some aspects, the condition is diabetes mellitus type 1, diabetes mellitus type 2 or a neurodegenerative disease, such as Alzheimer's disease, Parkinson's disease, epilepsy, dementia, schizophrenia, depression, anxiety or autism spectrum disorder.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the agent, the route of administration, etc. It is understood, however, that specific dose levels of the therapeutic agents of the present invention for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. In general, one will desire to administer an amount of the agent or compositions of this invention to modulate CDP138 activity in vivo to result in inhibition by at most 80% of the activity value in a control sample, or alternatively at most 60%, or alternatively at most 40%, or alternatively at most 20, or alternatively at most 10%, or in activation of CDP138 activity by 110%, optionally 450%, optionally 200-500%, or 1000-3000% higher as compared to control. Determination of these parameters is well within the skill of the art. These considerations, as well as effective formulations and administration procedures are well known in the art and are described in standard textbooks. In some aspects, the modulation of CDP138 activity is binding Ca²⁺, binding lipid membranes or inducing fusion of GLUT4 vesicles with a plasma membrane.

In some embodiments, the invention provides a method for inhibiting proliferation of a cancer cell by contacting the cancer cell with an agent that modulates CDP138 activity in the cancer cell, whereby increased activity of the CDP138 inhibits the cancer cell from dividing, thereby inhibiting proliferation of said cancer cell. Inhibition of proliferation as used herein includes, but is not limited to, preventing the cancer cell from dividing, induction of apoptosis or induction of cell death. Methods for measuring inhibition of cell proliferation are well known in the art and any number of these methods can be used in the methods of the invention. In some aspects, contacting the cell occurs in vitro or in vivo. In some aspects, the agent is a chemical compound, such as a small molecule, a nucleic acid or a protein.

In some aspects, the cancer cell originated from a tissue, such as, but not limited to, bone marrow, muscle, kidney, lung, colon, bladder, liver, pancreas, prostate, skin, breast, thyroid, saliva gland, ovary, lymph node and brain.

In some embodiments, the invention provides a method for inducing cell cycle arrest of a cancer cell by introducing into the cancer cell a nucleic acid molecule encoding CDP138 or an active fragment thereof, whereby expression of the CDP138 or active fragment thereof induces cell cycle arrest of the cancer cell. Inducing cell cycle arrest as used herein includes, but is not limited to, stopping the cell from progressing past one or more of the stages of the cell cycle, including G1, S, G2 or M phase. As exemplified herein, the formation of multiple nuclei in the cell is an example of the type of cellular phenotype associated with cycle arrest. In some aspects, contacting the cell occurs in vitro or in vivo.

In some aspects, the nucleic acid molecule encodes a polypeptide selected from, but not limited to, human CDP138, mouse CDP138, rat CDP138, chicken CDP138, dog CDP138 or chimpanzee CDP138. In some aspects, the CDP138 has a mutation or modification at a position in the amino acid sequence of CDP138, such as, but not limited to, S197, S260, S262, S295, T314, T330, S597, S652, S726, S855, D19, D26, D76, D78, D84 and the C2 domain of CDP138.

In some aspects, the cancer cell originated from a tissue selected from, but not limited to, bone marrow, muscle, kidney, lung, colon, bladder, liver, pancreas, prostate, skin, breast, thyroid, saliva gland, ovary, lymph node or brain.

Animal model systems can be useful for elucidating normal and pathological functions of CDP138. Accordingly, the invention provides CDP138-deficient non-human animals, or CDP138 “knock-out” animals, such as a transgenic mouse. Methods of deleting all or a portion of a gene so as to alter or prevent expression of the naturally occurring polypeptide are well known in the art. Gene knockout by homologous recombination is described, for example, in Houdebine, Methods Mol. Biol. 360:163-202 (2007), and in U.S. Pat. Nos. 5,616,491, 5,750,826, 5,981,830. One exemplary method of making and using an CDP138 knockout mouse are described herein in Example V. Analogous targeting vectors and methods are expected to be useful in generating CDP138 knockout animals.

Accordingly, in some embodiments, the invention provides a transgenic mouse whose genome comprises a null allele of the gene encoding CDP138. In some aspects of the invention, the transgenic mouse exhibits high-fat diet-induced insulin resistance, glucose intolerance, heart hypertrophy or fibrosis. In some aspects of the invention, the gene encoding CDP138 is 5730419I09Rik or a variant thereof. The invention also provides that the transgenic mouse can be heterozygous or homozygous for the null allele.

A “null allele” is a mutant copy of a gene that lacks or significantly inhibits that gene's normal function. This can be the result of the complete absence of the gene product (protein, RNA) at the molecular level, or the expression of a non-functional gene product. At the phenotypic level, a null allele is indistinguishable from a deletion of the entire locus. As disclosed here, on example of a null allele of the gene CDP138 can be an insert trap between the 1st and 2nd Exons of mouse 5730419I09Rik gene.

In some embodiments, the invention provides a method for identifying an agent that modulates insulin resistance, glucose intolerance, heart hypertrophy or fibrosis formation using a transgenic mouse of the invention. In some aspects of the invention, the method comprises administering a candidate agent to a transgenic mouse as disclosed herein, and determining a level of insulin resistance, glucose intolerance, heart hypertrophy or fibrosis formation in the transgenic mouse administered with the agent. The methods of the invention can also include comparing the level insulin resistance, glucose intolerance, heart hypertrophy or fibrosis formation in the transgenic mouse administered with the agent to a control level of insulin resistance, glucose intolerance, heart hypertrophy or fibrosis formation. It is understood that methods for determining and comparing the level of insulin resistance, glucose intolerance, heart hypertrophy or fibrosis formation are well known in the art. Moreover, non-limiting examples of such methods are disclosed herein in Example V. In some aspects of the invention, the control level can be predetermined by prior analysis or experimentation or is obtained from an untreated transgenic animal.

The agent that modulates insulin resistance, glucose intolerance, heart hypertrophy or fibrosis formation can be identified when altered levels of insulin resistance, glucose intolerance, heart hypertrophy or fibrosis formation are found in the treated transgenic animal, which indicates that the candidate agent modulates insulin resistance, glucose intolerance, heart hypertrophy or fibrosis formation. In some aspects of the invention, the candidate agent is selected from a chemical compound, a nucleic acid and a protein as disclosed herein.

The invention also provides for a method of treating diabetes, obesity or myocardial dysfunction in a patient in need thereof. The methods of the invention include administering a therapeutic amount of an agent that modulates the activity of CDP138. In some aspects, the activity of CDP138 can be increased or decreased. The increased or decreased activity can be relative to a predetermined level of activity of CDP138 or compared to a level of activity as determined from control cell, tissue or organ.

The activity of CDP138 can be modulated using methods well known to those of skill in the art, such as, but not limited to, increasing the expression of the gene encoding CDP138, or alternatively administering a pharmaceutical composition comprising isolated CDP138 protein or an fragment thereof as described herein, or alternatively administering an agent that has been identified using a method of the invention as an agent that modulates the activity of CDP138, such as, but not limited to, insulin resistance, glucose intolerance, heart hypertrophy or fibrosis formation.

The “therapeutically effective amount” will vary depending on the agent used, the disease and its severity and the age, weight, etc., of the patient to be treated, all of which is within the skill of the attending clinician. It is contemplated that a therapeutically effective amount of one or more agent identified using a method described herein will be one that alters the activity of CDP138 to an extent that results an improvement in one or more of the phenotypes disclosed herein. For example, the altered activity of CDP138 can impede or reduce insulin resistance, glucose intolerance, heart hypertrophy or fibrosis formation. Accordingly, a therapeutically effective amount of an agent of the invention can be used to treat diabetes, obesity or myocardial dysfunction.

It is understood that modifications which do not substantially affect the activity of the various embodiments of this invention are also provided within the definition of the invention provided herein. Accordingly, the following examples are intended to illustrate but not limit the present invention.

### Example I

### CDP138 is Required for Insertion of GLUT4 into the Plasma Membrane

In this study, quantitative phosphoproteomics and RNAi-based functional analyses was used to identify a previously uncharacterized 138 kDa C2 domain-containing phosphoprotein (CDP138) that is required for GSV-PM membrane fusion during GLUT4 translocation, and for subsequent glucose transport. The data presented herein shows that CDP138 functions downstream of Akt2 and dynamically associates with both the PM and GLUT4 vesicles. In addition, the C2 domain binds Ca²⁺ and membrane lipids. Both the intact C2 domain and the Akt2 phosphorylation site of CDP138 are necessary for insulin-stimulated GLUT4 translocation. These observations establish CDP138 as a novel molecular link between insulin-stimulated Akt2 signaling and GLUT4 insertion into the PM.

The protein kinase Bβ (Akt2) pathway is known to mediate insulin-stimulated glucose transport through increasing glucose transporter GLUT4 translocation from intracellular stores to the plasma membrane (PM). Combining quantitative phosphoproteomics with RNAi-based functional analyses, we show that a previously uncharacterized 138-kDa C2 domain-containing phosphoprotein (CDP138) is a substrate for Akt2, and is required for insulin-stimulated glucose transport, GLUT4 translocation, and fusion of GLUT4 vesicles with the PM in live adipocytes. The purified C2 domain is capable of binding Ca²⁺ and lipid membranes. CDP138 mutants lacking the Ca²⁺-binding sites in C2 domain or Akt2 phosphorylation site Ser197 inhibit insulin-stimulated GLUT4 insertion into the PM, a rate-limiting step of GLUT4 translocation. Interestingly, CDP138 is dynamically associated with both GLUT4 vesicles and the PM in response to insulin stimulation. Together, these results suggest that CDP138 is a key molecule linking the Akt2 pathway to the regulation of GLUT4 vesicle-PM fusion.

Highlights of the experimental results include: 1) CDP138 is required for insulin-stimulated GLUT4 vesicle-PM fusion; 2) Akt2 phosphorylates CDP138 at Ser197 residue, which is critical for GLUT4-PM fusion; 3) Ca²⁺-binding sites in the C2 domain are required for membrane interaction and GLUT4 translocation; and 4) CDP138 is dynamically associated with GLUT4 vesicles and the PM in insulin-stimulated cells.

DNA constructs, siRNAs, and antibodies. Constitutively active HA-myr-Akt2 in the pcDNA3 expression vector was from Addgene (Cambridge, Mass.). IRAP-pHluorin and GLUT4-EGFP were kindly provided by Dr. Tao Xu (Institute of Biophysics, Beijing, China). The pCMV5 plasmid DNA encoding Myc-GLUT4-GFP was constructed as described (Jiang et al., 2002). The cDNA clone of human KIAA0528 (accession number BC117143) was obtained from the ATCC (#11048509). To make a full-length CDP138 tagged with a HA epitope at the N-terminus and mCherry at the C-terminus, CDP138 cDNA was cloned into the HA-tagged pCMV5 and pcDNA3-mCherry vectors, respectively, as described herein. The mutants of HA-CDP138 and CDP138-mCherry, including ΔC2, 5DA (five aspartate residues converted to alanine), Ser197Ala and Ser200Ala were made by PCR-based mutation. SiRNA duplexes against mouse 5730419I09RIK and Akt2 were from Dharmacon Research, Inc. For detailed cloning strategies and siRNA sequences see description herein. Antibodies used for immunoprecipitation and immunoblotting are described earlier (Zhou et al., 2010) and herein.

Cell culture, siRNA and gene transfection, and SILAC medium. The 3T3-L1 fibroblasts were grown in DMEM supplemented with 10% FBS, 50 μg/ml streptomycin, and 50 unit/ml penicillin. The fibroblasts were differentiated into adipocytes, and then transfected with siRNA duplexes or DNA constructs by electroporation with a Bio-Rad Gene Pulser Xcell™ system as described (Jiang et al., 2003). Oligofectamin-2000™ reagents (Invitrogen) for transfection of plasmid DNA into CHO-T and HEK293T cells. For SILAC experiments, the DMEM containing Arg-0/Lys-H4, Arg-6/Lys-D4, and Arg-10/Lys-8 (Silantes, GmbH) was used as described herein.

Mass spectrometric analysis. After SDS/PAGE, in-gel trypsin digestion, and peptide extraction, samples were processed for liquid chromatography-mass spectrometry. A detailed description of the mass spectrometric measurements can be found herein.

CDP138 C2 domain purification, calcium and lipid membrane binding assays. The WT and 5DA sequences of 354 bp were cloned between BamHI and HindIII of the pMal-C2 vector (Amersham). MBP-WT1-118 and MBP-5DA1-118 were expressed after induction at 16° C. for 20 h with 1 mM isopropyl-β-d-thiogalactopyranoside (IPTG) in BL21 bacteria. MBP fusion proteins were subjected to amylose affinity chromatography then further purified on a Superdex 200 size exclusion column with an Äkta FPLC system (GE Healthscience) as described in herein. Binding of Ca²⁺ to the C2 domain fusion proteins was measured by tryptophan fluorescence spectra as described in detail in herein. Binding of C2 domain fusion proteins to lipid membranes was determined by fluorescence resonance energy transfer (RET) as described earlier (Qin et al., 2004).

Deoxyglucose uptake assay. To detect the effect of specific gene silencing on insulin-stimulated glucose transport, [3H]-deoxyglucose uptake assays were carried out in 3T3-L1 adipocytes as described earlier (Jiang et al., 2003) and herein.

In vitro protein kinase assay. For the in vitro Akt2 kinase assays, constitutively active HA-myr-Akt2 (with a PH domain deletion) and HA-CDP138-WT were expressed in HEK293T cells and immunoprecipitated using the anti-HA Ab (clone F3, Roach) from 2 mg total lysate in HEPES buffer with 10 mM CHAPS. The kinase assay was performed on aliquots of the immunoprecipitate in 100 μl of reaction mixture consisting of 20 mM MOPS (pH 7.5), 75 mM MgCl2, 1 mM dithiothreitol (DTT), 5 mM sodium orthovanadate, 100 μM ATP, 10 μCi γ-³²P-ATP, 5 mM β-glycerophosphate and 1 mM EGTA for 20 min at 30° C.

Isolation of GLUT4-containing vesicles and subcellular fractionation. Serum-starved 3T3-L1 adipocytes were stimulated with or without 100 nM insulin for 10 min or 30 min. GLUT4 vesicles were enriched by immunoabsorption with anti-GLUT4 Ab (1F8) from adipocytes after removal of nuclear and the PM fractions as previously described (Kandror and Pilch, 2006). Post-nuclear subcellular fractionation was performed by ultracentrifugation with continuous iodixanol gradients as described (Chen et al., 2007). Detailed procedures are described herein.

GLUT4 translocation detection with wide field fluorescence and TIRF microscopy: Both wide field and TIRF microscopy were used to detect myc-GLUT4-GFP translocation to the cell surface as previously described (Jiang et al., 2002) and herein.

Analysis of GLUT4 trafficking and GSV-PM fusion in live cells with TIRF microscopy. Differentiated adipocytes were transfected by electroporation with the plasmid DNA encoding IRAP-pHluorin or GLUT4-EGFP and siRNAs, or pcDNA3-mCherry constructs encoding CDP138 fusion protein and its mutants. Cells were reseeded to grow in 35 mm glass-bottom microwell dishes (MatTak Corp, Ashland, Mass.). Live cell imaging was performed to measure membrane fusion and GLUT4 trafficking with IRAP-pHluorin and GLUT4-EGFP as molecular probes, respectively, using a Nikon Eclipse-Ti TIRF microscope equipped with perfect focus system and multiple points capture program as described herein.

Statistics. For all the quantified data, population averages are given as mean and standard deviation (SD) or standard error of the mean (SEM). Statistical significance was tested using unpaired two-tailed Student's t-test.

Antibodies and Procedures for Immunoprecipitation and Immunoblotting Antibodies: The monoclonal Ab against HA (haemagglutinin, HA.11, clone 16B12) was from Covance, Dedham, Mass. High affinity rat anti-HA (F3) Ab for immunoprecipitation was from Roche Applied Science, Mannheim, Germany. Monoclonal and polyclonal Ab against Myc epitope and rabbit polyclonal Ab against phospho-Akt (Ser473), the phosphorylated Akt substrate motif (lot 2 & 5), Erk1/2, and IRS-1 were from Cell Signaling Technology, Inc., MA. Polyclonal antibody for β-actin was from Sigma. The mAb against GLUT4 (1F8) was from AbD Serotec, and the goat anti-GLUT4 pAb was from Santa Cruz Biotechnology. Affinity purified rabbit pAb against peptide (Acetyl-TKPHVEKSLQRASTDNEELC-amide) of kiaa0528 were produced by Bethyl Laboratories, Inc. (Montgomery, Tex.).

Procedures: After experimental treatments, the cells were solubilized in ice cold lysis buffer containing 50 mM Hepes (pH 7.4), 137 mM NaCl, 5 mM sodium pyrophosphate, 5 mM β-glycerophosphate, 10 mM sodium fluoride, 2 mM EDTA, 2 mM Na3VO4, 1 mM PMSF, 10 μg/ml aprotinin, 10 μg/ml leupeptin and 1% Triton X-100. Total cell lysates of 1-2 mg of protein were immunoprecipitated with antibodies against phospho-Akt/PKB substrates (1:100 dilution, lots 2 & 5, Cell Signalling, Inc), Myc epitope (9E10) or HA epitope (F3, Roach) for 2 h followed by incubation with 80 μl of Protein A-Sepharose 6 MB (GE Healthcare) for 2 h at 4° C. The beads were then washed four times with lysis buffer before boiling for 5 min in Laemmli buffer. To detect the phospho-Akt/PKB substrates phospho-Ser473 Akt/PKB and phospho-Thr202/Tyr204 Erk1/2, total cell lysates (20 to 50 μg of protein) were resolved with SDS/PAGE and electrotransferred to nitrocellulose membranes. Membranes were incubated with rabbit polyclonal antiphospho-specific antibodies (1:1000 dilution) overnight at 4° C. The following antibodies were used to detect their antigens using 20 to 50 μg of proteins from total cell lysates: rabbit anti-total Akt/PKB pAb (1:1000 dilution), rabbit anti-phosph-S473-Akt antibody, rabbit anti-CDP138 pAb (1 μg/ml), mouse anti-Myc epitope mAb (1:1000), anti-HA epitope mAb (1:1000), and anti-GLUT4 mAb (1:1000). All membranes were then incubated with the appropriate horseradish peroxidase-linked secondary antibodies (1:10000 dilution each) for 1 h at room temperature. The membranes were washed (PBS, pH 7.4, 0.1% Tween 20) for 1 h at room temperature after incubation with each Ab, then detected with ECL® (enhanced chemiluminescence) kit.

DNA Constructs and siRNAs. A full-length cDNA clone of human KIAA0528 (accession number BC117143: GI: 109658767) was obtained from the ATCC (#11048509) with IMAGE clone ID 40125694. The clone was provided in a pCR4-TOPO vector. To make the full-length CDP138 tagged with 3HA epitope at the Nterminus, CDP138 cDNA was cloned in flame downstream from the 3HA-tagged coding sequence in pCMV5 vector with restriction enzymes Mlu1 and BamHI. To express full-length CDP138 fused with mCherry at its C-terminus, the stop codon before the BamH1 site was removed using PCR-based mutagenesis, and the cDNA ligated into the pcDNA3-mCherry vector at the Mlu1 and BamHI sites. The mutants of 3HA-CDP138 and CDP138-mCherry, including delta C2, 5DA (five aspartate residues converted to alanine), Ser197Ala were made by the PCR-based site-directed mutagenesis are described below and the DNA sequences for all expression constructs were confirmed before using in the expression studies.

To make 3HA-CDP138-ΔC2 expression vector, the first 405 nucleotides were removed from the 5′ end of CDP138 cDNA using Mlu1 (MCS site) and Cla1 (405) restriction enzymes and a polylinker was used to ligate the remaining cDNA in right flame to the pCMV5-3HA vector.

3HA-CDP138-5DA mutant was generated by converting the aspartate residues in loop 1 (D19A, D26A) and in loop 3 (D76A, D78A, D84A) into alanine residues by PCR-directed mutagenesis. Two rounds of PCR reactions were carried out using two pairs of PCR primers to introduce the mutation in both loop 1 and loop 2 with the PCR products including both Mlu1 and Cla1 restriction sites before replacing the wild type CDP138 with the PCR product.

To convert Ser179 to Ala or Ser200 to Ala, PCR primers were used to amplify the Ser197Ala containing fragment including the Cla1 (405) and HindIII (1093) sites. Then the PCR fragment was inserted into Cla1 and HindIII-digested pcMV5-3HA-CDP138-WT.

To construct an expression vector for CDP138 with mCherry fused to its C-terminus, a PCR reaction was used to remove the stop codon before the BamH1 cloning site in pCMV5-3HACDP138-WT vector. Then Ser197Ala, 5DA, and double mutants were ligated into the pCMV5-3HA-CDP138-WT vector without a stop codon before cutting with Kpn1 (MCS) and BamH1. Finally, the fragments were inserted into a modified pmCherry-N3 vector, kindly provided by Dr. John Reed (Sanford-Burnham Medical Research Institute), to produce expression vectors with mCherry in flame at the C-terminus. All DNA constructs were sequenced to confirm the mutation sites and total cell lysates from transfected HEK293T cells were used immunoblotting to validate protein expression.

siRNA duplexes against mouse 5730419I09RIK and Akt2 are from Dharmacon Research, Inc. The smart pool siRNAs targeting sequences for mouse 5730419I09RIK (NM_{—}001109688) are as follows: GCAAGGUUAUGUCGAUUAA; GGCCACAGGAGUCUACUUA; GUAAGAGUGGUCAGACUAA and AUACAGAAAUUAUGCCUGG. SiRNAs duplexes targeting sequences for mouse Akt2 are GAGAGGACCUUCCAUGUAG and UGCCAUUCUACAACCAGGA.

The scrambled siRNAs duplexes are CAGUCGCGUUUGCGACUGGdTdT and dTdTGUCAGCGCAAACGCUGACC.

Purification of MBP Fusion Protein The WT and 5DA sequence of 354 bp were amplified by PCR from pcmv5-WT and pcmv5-5DA with primers containing BamHI and Hind III restriction sites. After digestion and ligation of the PCR products and pMal-C2 vector (Amersham), a transformation into BL21 bacteria was performed. After screening, positive clones were verified by sequencing. Expression of MBPWT1-118 and MBP-5DA1-118 were induced at 16° C. for 20 h with 1 mM isopropyl-β-Dthiogalactopyranoside (IPTG) in BL21 bacteria. The culture was harvested by centrifugation at 2000g, resuspended in PBS (0.14 M NaCl, 2.7 mM KCl, 10.1 mM Na₂HPO₄, and 1.8 mM KH₂PO₄, pH 7.3) containing a mix of protease inhibitors consisting of 1 μM aprotinin, 10 μM leupeptin, 1 μM pepstatin, 5 mM benzamidine and 1 mM PMSF. After sonication for 6 cycles of 10 sec, power 7.5, a centrifugation at 8000g 30 min 4° C. was performed and the supernatants containing the MBP fusion proteins were rotated with amylose beads (New England Biolabs) for 1 h at 4° C. The beads were washed with 10 bead volumes, and the fusion proteins were eluted in PBS buffer containing maltose 10 mM (New England Biolabs). Finally, recombinant MBP fusion proteins were applied to a Superdex 200 gel exclusion column and purified using the Äkta FPLC system (GE Healthscience). The purity of fusion proteins was assessed using SDS gel electrophoresis, and protein was quantified using the BCA assay.

Deoxyglucose Uptake Assay To detect the effect of specific gene silencing on insulin-stimulated glucose transport, [3H]-deoxyglucose uptake assays were carried out in 3T3-L1 adipocytes as described previously (Jiang et al, 2003). Briefly, siRNA-transfected cells were reseeded on 24-well plates and cultured for 72 h before serum starvation for 3 h with KRH (Krebs-ringer Hepes) buffer (130 mM NaCl, 5 mM KCl, 1.3 mM CaCl2, 1.3 mM MgSO4, 25 mM HEPES, pH 7.4) supplemented with 0.5% BSA and 2 mM sodium pyruvate. Cells were then stimulated with insulin for 30 min at 37° C. Glucose uptake was initiated by addition of 100 μM [1, 2-3H]2-deoxy-D-glucose to a final assay concentration for 5 min at 37° C. Assays were terminated by four washes with ice-cold KRH buffer, the cells were solubilized with 0.4 ml of 1% Triton X-100, and radioactivity was determined by scintillation counting. Non-specific deoxyglucose uptake was measured in the presence of 20 μM cytochalasin B and was subtracted from each determination.

Isolation of GLUT4-containing Vesicles Serum-starved 3T3-L1 adipocytes were stimulated with or without of 100 nM insulin for 10 min or 30 min. GLUT4 vesicles were enriched by immunoabsorption with anti-GLUT4 Ab (1F8) from adipocytes after removal of nuclear and the PM fractions as previously described (Kandror and Pilch, 2006). The cells were then washed, scraped into cold PBS, and spun at 1000g for 5 min. The cell pellet was suspended in PBS containing 1 mM PMSF, 5 mM NaF, 1 mM sodium pyrophosphate, 10 mM Na3VO4, 1 mM β-glycerophosphate, 5 mM benzamidine, and 10 μg/ml leupeptin and aprotinin. Cells were disrupted by passing 12 times through a 26 G needle and nuclei were pelleted twice at 800g for 10 min at 4° C. The isolation of GLUT4 storage vesicles was performed as previously described (Kandror and Pilch, 2006). The post-nuclear supernatant was collected and centrifuged for 30 min at 16,000g, at 4° C. The supernatant containing light membranes and cytosol was immunoabsorbed for 12 hours at 4° C. with monoclonal anti-GLUT4 Ab (1F8) covalently immobilized on protein A/G beads. After four washes with PBS, GLUT4 vesicle-associated proteins were eluted with 1% Triton in PBS, followed by solubilization of IgG-bound GLUT4 with Laemmli buffer, in the absence of reducing agents.

Subcellular Fractionation Serum-starved 3T3-L1 adipocytes were stimulated with 100 nM insulin for 10 min or 30 min. Washed cells were homogenized in homogenization buffer (20 mM Tris pH 7.5, 255 mM sucrose, 1 mM EDTA, 1 mM PMSF, 5 mM NaF, 1 mM sodium pyrophosphate, 10 mM Na3VO4, 1 mM β-glycerophosphate, 5 mM benzamidine, 10 μg/ml leupeptin and aprotinin). Cells were disrupted with a 26 G needle and nuclei were pelleted twice at 800g for 10 min at 4° C. Subcellular fractionation was performed by mixing the post-nuclear supernatant with 60% optiprep and homogenization buffer to generate 10, 20 and 30% iodixanol solutions as previously described (Chen et al., 2007). Equal volumes of these three solutions were layered in SW60Ti centrifuge tubes and samples were centrifuged at 260,000g for 3 h at 4° C. with the brake off. Fractions were collected from the top of the tubes, mixed with Laemmli buffer, and boiled.

Preparation of the Triple-Labeling SILAC Media. Arginine- and lysine-free DMEM was divided into three equal portions and 28 mg/liter of the three Arginine isotopes were added separately to make the Arg-0, Arg-6, and Arg-10 media, respectively. In addition, 48.7 mg/liter L-lysine, L-lysine-D4, and L-lysine13C6, 15N2 were supplemented separately to the three lots containing Arg-0, Arg-6, and Arg-10. Finally, glutamine and antibiotics were added to the media together with the full complement of amino acids (Arg-0/Lys-H4, Arg-6/Lys-D4, and Arg-10/Lys-8) and then sterile-filtered (Millipore). The sterile media were separated into two portions for dialyzed serum-containing and serum free media.

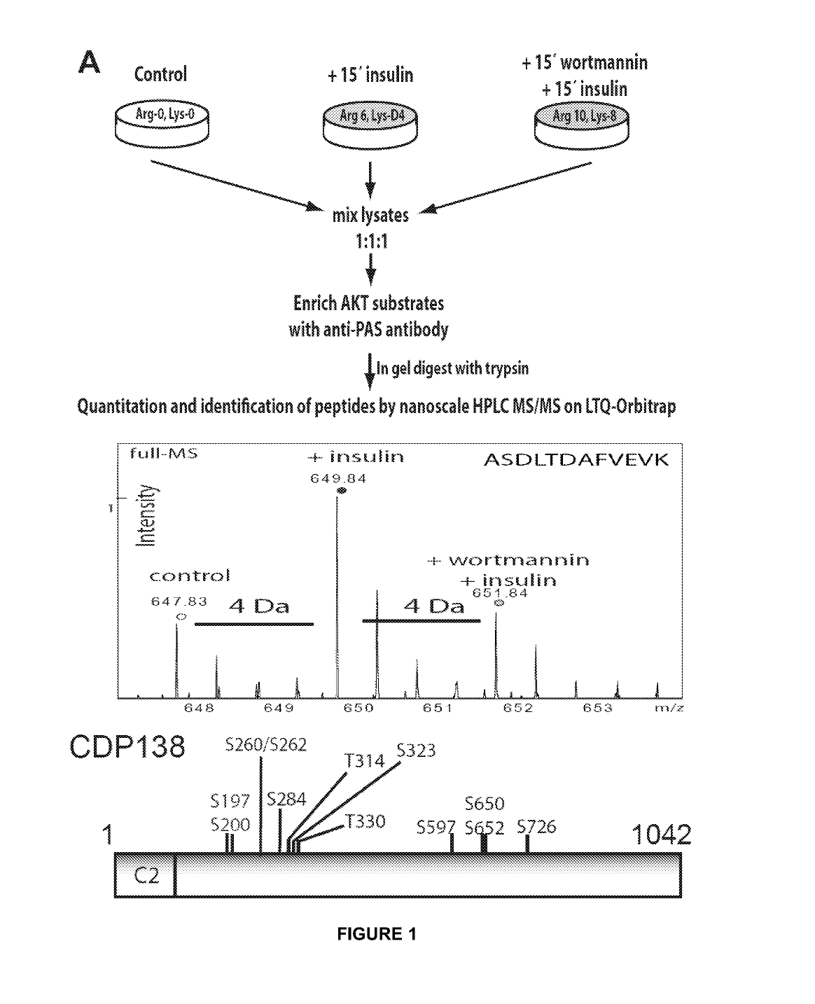

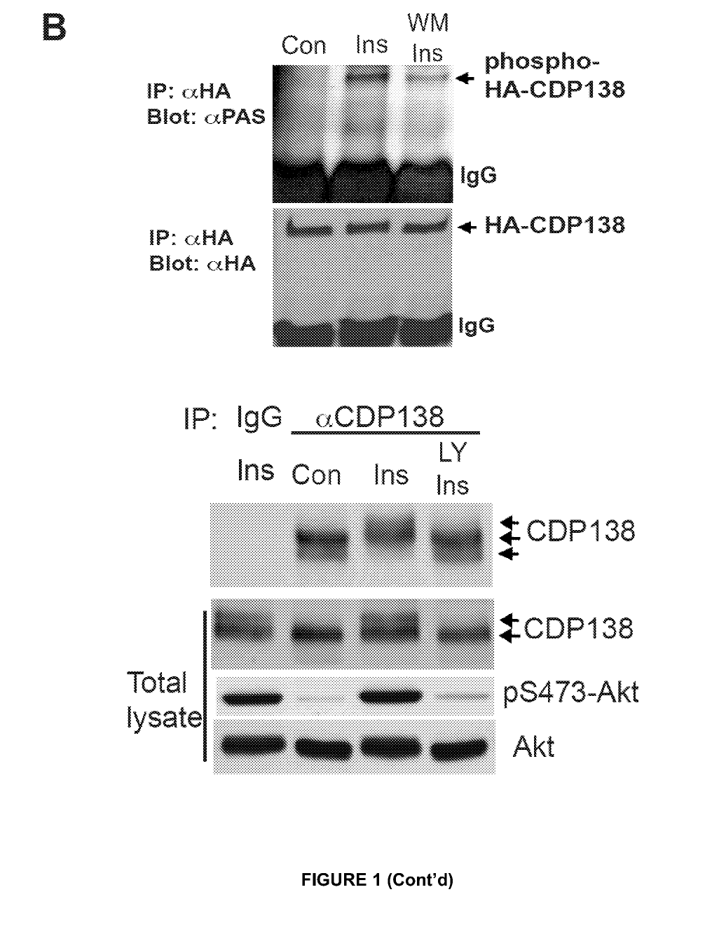

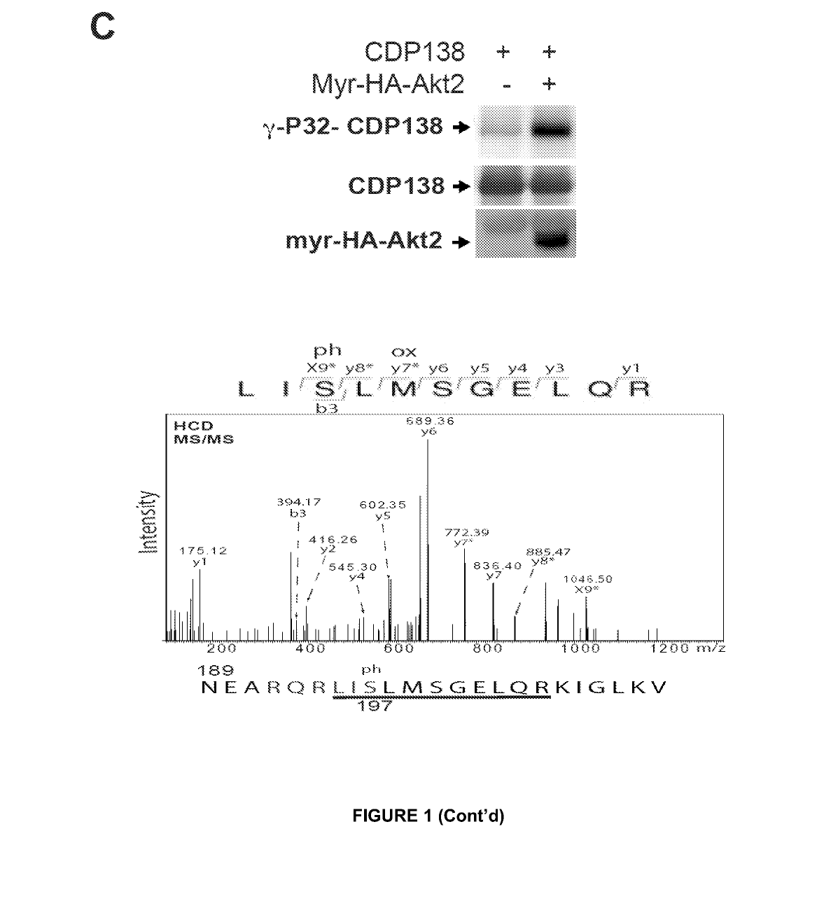

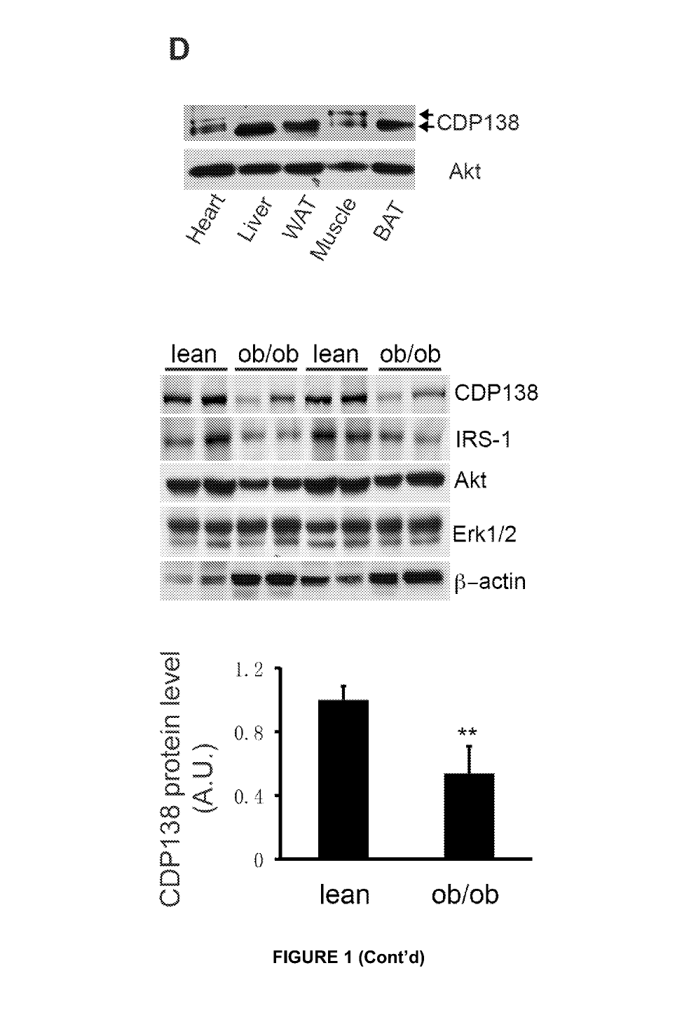

Mass Spectrometric Analysis. For SILAC quantitative proteomic study, the mixture of total lysates from the 3 different groups (as shown in FIG. 1A) was used for immunoprecipitation with PAS antibodies. Enriched proteins were resolved by SDS-PAGE. After staining of the gel with the Colloidal Blue Staining Kit (Invitrogen), excised gel pieces were subjected to in-gel reduction and alkylation, followed by trypsin digestion as described previously (Shevchenko et al., 2007). Finally, peptides were extracted by adding an equal volume of 30% acetonitrile/0.3% trifluoroacetic acid (TFA), followed by a final extraction with 100% acetonitrile. Extracts were evaporated in a Speedvac to remove acetonitrile and subsequently acidified with 0.5% TFA. Samples were desalted and concentrated with Stop and Go extraction tips (STAGE tips) (Rappsilber et al., 2007). Reverse phase nano-LC-MS/MS was performed with an Agilent 1200 nanoflow LC system (Agilent technologies) using a cooled thermostated 96-well autosampler. The LC system was coupled to a LTQ-Orbitrap instrument (Thermo Electron) equipped with a nanoeletrospray source (Proxeon). Chromatographic separation of peptides was performed in a 10-cm long 8-mm tip opening/75-mm inner diameter capillary needle filled with reverse-phase ReproSil-Pur C18-AQ 3-mm resin. The mass spectrometers were operated in the data-dependent mode to automatically measure MS, MS/MS, and MS3 spectra LTQ-Orbitrap full scan MS spectra were acquired with a resolution r=50,000 at m/z 400. The following search parameters were used in all MASCOT searches: maximum of 2 missed trypsin cleavages, cysteine carbamidomethylation, methionine oxidation, pSTY, N-term protein acetyl, SILAC labels: Lys-D4, Lys-8, Arg-6, and Arg-10. The maximum error tolerance for MS scans was 10 ppm and 0.5 Da for MS/MS. Only proteins that had at least 2 ion scores >20 were identified and quantified. MaxQuant was used to verify and quantify the resulting peptide pairs.

For the CDP138 immunoprecipitation studies, enriched proteins were separated by one-dimensional SDS-PAGE and stained with colloidal Coomassie. CDP138 gel bands were excised and subjected to in-gel digestion with trypsin. The resulting tryptic peptides were extracted, and desalted as described above. In-solution digests were performed. Mass spectrometric experiments were performed on a nano-flow HPLC system (Proxeon) connected to a LTQorbitrap Velo instrument (Thermo Fisher Scientific) equipped with a nanoelectrospray source (Proxeon). The mass spectrometer was operated in the data dependent mode to monitor MS and MS/MS spectra. Survey full-scan MS spectra (from m/z 300-2000) were acquired in the orbitrap with a resolution of R=60,000 at m/z 400 after accumulation of 1,000,000 ions. The 15 most intense ions from the preview survey scan delivered by the orbitrap were sequenced by collision-induced dissociation (CID) in the LTQ. For higher C-trap dissociation (HCD) 30,000 ions were accumulated at 400 m/z in the c-trap and MS/MS spectra from the 10 most intense ions were detected in the orbitrap at a resolution of 7500 (Olsen et al., 2009). Mass spectra were analyzed using MaxQuant software (Cox et al., 2008) and automated database searching (Matrix Science). All tandem mass spectra were searched against the mouse International Protein Index protein sequence database (IPI version 3.54) and concatenated with reversed copies of all sequences. The required false positive rate was set to 1% at the protein level, and maximum allowed mass deviation was set to 7 ppm in MS mode and 0.5 Da for MS/MS peaks. Cysteine carbamidomethylation was searched as a fixed modification and N-acetyl protein, oxidized methionine, acetylation of lysine, and phospho-STY, was searched as variable modifications. A maximum of three missed cleavages were allowed.

Immunofluorescence Microscopy, Total Internal Reflection Fluorescence (TIRF) Microscopy, Image Analysis, and Quantification. Immunofluorescence Microscopy. To detect HA-tagged CDP138 expression and endogenous Akt phosphorylation, adipocytes were fixed with 4% formaldehyde, washed with PBS and permeabilized with PBS containing 1% FBS and 0.5% Triton X-100. Cells were then incubated with primary rabbit mouse anti-HA, or rabbit anti-pSer473-Akt antibodies overnight at 4° C. After washing, cells were incubated with Alexa Fluor 568-labeled goat anti-mouse IgG or anti-rabbit IgG for 30 min at room temperature.

To test the effect of RNAi-induced gene specific knockdown of CDP138 or overexpression of CDP138 mutants on GLUT4 translocation, siRNAs or CDP138 constructs were transfected into adipocytes together with myc-GLUT4-GFP by electroporation as previously described (Jiang et al, 2003). Cell were reseeded for 48 hr (for the overexpression study) or 72 hr (for the siRNA study), serum starved for overnight then treated with or without insulin for 20 min. Cells were washed with cold PBS, fixed with 4% formaldehyde and immunostained. Briefly, for siRNA study, the cell surface Myc-GLUT4-GFP was visualized with mouse anti-Myc Ab (9E10) and AlexaFluoro568-labeled goat anti-mouse secondary Ab (Invitrogen) in PBS containing 1% FBS. After washing, the coverslip were mounted with Prolong antifade reagent (Invitrogen, Eugene Oreg.). Fluorescence microscopy was carried as described below. Then, images were acquired with wide field microscopy as described below. For the study with overexpressed HAtagged CDP138 constructs, the cell surface Myc-GLUT4-GFP was visualized with rabbit anti-Myc Ab (Cell signaling, Inc) and AlexaFluoro568-labeled goat anti-rabbit secondary Ab (InVitrogen) in PBS containing 1% FBS. Cells were further permeabilized with PSB containing 0.5% Tween-20, and HA-tagged protein was detected with a mouse anti-HA epitope primary Ab followed by AlexaFluoro-350-labelled goat anti-mouse IgG. Insulin-induced myc-GLUT4 accumulation (AlexaFluoro-568 signal) within the evanescence excitation field (100 nm from dorsal membrane) in transfected adipocytes was measured with a Nikon Eclipse-Ti microscope equipped with a CoolSNAP HQ2 camera and laser scanning TIRF capacity. Total GLUT4-GFP signals and FIA-CDP138 Alexa Fluoro-350 signals were acquired with wide field imaging to identify double-transfected cells. The ratio of myc-GLUT4 signal in the TIRF zone to the total Epi-GFP signal in wide field was used as a measure of GLUT4 translocation to the cell surface.

Total Internal Reflection Fluorescence (TIRF) Microscopy: Nikon (Melville, N.Y.) TI-TIRF-E, which was based on the objective lens method, was used to perform all the TIRF experiments. For both live and fixed cell imaging in this study, an Apo TIRF 60×1.49 numerical aperture oil immersion objective was used. The penetration depth of the evanescent field was estimated to be around 100 nm. For live cell imaging, differentiated adipocytes were transfected with plasmid DNAs encoding IRAP-pHlorin or GLUT4-EGFP by electroporation and cells were reseeded to grow in 35 mm glass-bottom microwell dishes (MatTak Corp, Ashland, Mass.). Forty-eight hours after reseeding, adipocytes were serum starved in DMEM for 2 hr and then incubated in KRBH (Krebs-ringer bicarbonate HEPES) buffer (in mM: 129 NaCl, 4.7 KCl, 1.2 KH₂PO₄, 5 NaHCO₃, 10 HEPES, 3 glucose, 2.5 CaCl₂, 1.2 MgCl₂, 0.1% BSA (pH 7.2)) as described Jiang et al (2008) for live-cell imaging at 37° C. with insulin stimulation at 100 nM. Imaging was performed in a Tokai Hit incubation system (Tokai Hit Co, Japan) with the microscope platform maintained at 37° C. Positively transfected adipocytes were identified, and the insulin-stimulated IRAP vesicle-PM fusion events or GLUT4-EGFP trafficking to the TIRF zone were recorded every 3 min for a 30 min period using Nikon Eclipse-Ti TIRF microscope. During live cell image recording, perfect focus system and multiple points capture program (NIS Element) were used to acquire images from multiple positively transfected cells at each time point.

Image Analysis and Quantification: TIRF and wide field fluorescence microscopy were performed using a Nikon inverted microscope (Melville, N.Y.) with a cooled charge-coupled device camera. Both the TIRF and Wide field Images were collected with a 60×1.49 numerical aperture oil-immersion objective. A Nikon Element was used for subsequent image analysis and quantification. For wide field fluorescence microscopy, cells expressing Myc-GLUT4-EGFP were selected manually based upon green fluorescent protein (GFP) fluorescence. The sum intensity of ROI (Region of Interest) for both Myc and GFP fluorescence was exported after the background fluorescence was subtracted with the Element software. The Myc/GFP ratio was calculated for each cell and averaged over multiple cells for each experiment. For TIRF microscopy with fixed cells, the ratio of TIRF Myc/Epi-GFP was acquired by dividing the sum ROI intensity of TIRF Myc by that of the wide field GLUT4-GFP. Similarly, the ratio of TIRF GFP/Epi-GFP was acquired by dividing the sum ROI intensity of TIRF GFP by that of the wide field GLUT4-GFP. For TIRF live cell images, the fluorescence from different frames was normalized to the first frame value (before insulin stimulation) of the same cell. For confocal images, images were captured with an Apo TIRF 60× oil-immersion objective (N.A. 1.49). Element software was also used to take images and the final pictures were assembled using Adobe Photoshop and Illustrator.

Calcium Binding Assay Binding of Ca²⁺ to the proteins was measured by fluorescence spectroscopy. Protein solutions were prepared at a final concentration of 1.11 to 1.36 μM in a buffer of 65 mM NaCl, 0.1 mM EGTA, 7 mM Na-phosphate, 15 mM Tris-HCl, pH 7.2. The sample was placed in a 4×4 mm2 rectangular quartz cuvette and equilibrated at 37° C. in a J-810 spectrofluoropolarimeter (Jasco, Inc., Tokyo, Japan). Tryptophan fluorescence spectra were measured between 300 and 400 nm, using excitation and emission slit bandwidths of 2 and 10 nm, respectively, with an excitation wavelength of 290 nm. The protein sample was titrated with increasing concentrations of CaCl₂, followed by fluorescence measurement after each addition. Free Ca²⁺ concentrations were determined by using EGTA activity coefficient of 0.961 and an EGTA-Ca²⁺ dissociation constant of 107.9 nM at pH 7.2 and 37° C. (Bers et al., 1994). In negative control experiments, the protein sample was titrated with similar volumes of a calcium-free buffer. Ca²⁺-induced changes in tryptophan fluorescence intensity at 340 nm were corrected for changes due to the blank buffer and normalized as (F−F₀)/F₀, where F and F₀ are the fluorescence intensities at 340 nm in the presence and absence of Ca²⁺, respectively. Values of (F−F₀)/F₀ were plotted as a function of free Ca²⁺ concentration and fitted with a binding isotherm, as follows. Ca²⁺ binding to the protein was described as a bimolecular process:

P_{f}+Ca_{f} [symbol] Ca_{b} (1)

where P_{f}, Ca_{f}, and Ca_{b} stand for free protein, free calcium, and bound calcium, respectively. The dissociation constant is

$${K}_{D} = \frac{[ {P}_{f} ] [ {Ca}_{f} ]}{[ {Ca}_{b} ]} \qquad ( 2 )$$

If n Ca²⁺ ions bind to the protein with same dissociation constant K_{D}, then [Ca_{b}]=n[P_{b}], [P_{f}]=[Pₜ]−[P_{b}], and [Ca_{f}]=[Caₜ]−n[P_{b}], where the subscripts b, f, and t indicate bound, free, and total concentrations of the respective species. If there is a Ca²⁺-induced change in protein fluorescence, due to changes in the protein structure and hence tryptophan microenvironment, then this change will be proportional to the fraction of Ca²⁺-bound protein, i.e.

$$\frac{[ {P}_{b} ]}{[ {P}_{t} ]} = \frac{Δ F}{Δ {F}_{max}} \qquad ( 3 )$$

where ΔF=(F−F₀)/F₀ (see above), and ΔFmax is the maximum value of ΔF at saturation of Ca²⁺ binding. Incorporation of the above relationships into Eq. (2) yields the following expression for K_{D}:

$${K}_{D} = \frac{( [ {P}_{t} ] - \frac{Δ F}{Δ {F}_{max}} ⌈ {P}_{t} ⌉ ) ( [ {Ca}_{t} ] - n \frac{Δ F}{Δ {F}_{max}} ⌈ {P}_{t} ⌉ )}{n \frac{Δ F}{Δ {F}_{max}} ⌈ {P}_{t} ⌉} \qquad ( 4 )$$

Equation (4) can be converted into a quadratic equation with respect to ΔF and solved as follows:

$$Δ F = Δ {F}_{max} ( \frac{a}{2} \pm \sqrt{\frac{{a}^{2}}{4} - \frac{[ {Ca}_{t} ]}{n [ {P}_{t} ]}} ) \\ where \\ a ≡ 1 + \frac{{K}_{D}}{[ {P}_{t} ]} + \frac{[ {Ca}_{t} ]}{n [ {P}_{t} ]} \qquad ( 5 )$$

Only the negative sign in front of the square root was used because the positive sign produces physically meaningless values of ΔF/ΔFₘₐₓ>1. Furthermore, if the protein has two or more binding sites each of which is characterized with a distinct dissociation constant, then the total ΔF is the sum of components defined by Eq. (5), with corresponding ΔFₘₐₓ) K_{D}, and n. These parameters have been determined from the best fit of data with simulated binding isotherms, i.e. dependences of ΔF on [Caₜ].

Determination of Protein Binding to Lipid Membranes Protein binding to lipid membranes was determined by fluorescence resonance energy transfer (RET), as described previously (Qin et al., 2004). Briefly, lipids were dissolved in chloroform and mixed at desired proportions (see below). The solvent was evaporated under a stream of nitrogen, followed by desiccation for 3 h and suspension by vortexing in an aqueous buffer of 100 mM NaCl, 0.1 mM CaCl₂, 20 mM Tris-HCl, pH 7.2. Unilamellar lipid vesicles were prepared by extrusion of the lipid suspension through 100 nm pore-size polycarbonate membranes using a Liposofast extruder (Avestin, Ottawa, Canada). The lipid composition of membranes was selected to mimic the inner face of plasma membranes of mammalian blood cells (Hanahan, 1997; Boon and Smith, 2002), as follows: 12% 1-palmitoyl-2-linoleoyl-phosphatidylcholine (PLPC), 15% 1-palmitoyl-2-linoleoyl-phosphatidylethanolamine (PLPE), 10% 1-stearoyl-2-linoleoyl-phosphatidylethanolamine (SLPE), 5% 1-stearoyl-2-arachidonoylphosphatidylethanolamine (SAPE), 20% 1-palmitoyl-2-linoleoyl-phosphatidylserine (PLPS), 5% 1,2-dioleoyl-phosphatidylinositol (DOPI), 1% phosphatidylinositol-4-phosphate (PIP, from bovine brain), 1% 1,2-dioleoyl-phosphatidylinositol-4,5-bis-phosphate (PIP2), 4% sphingomyelin (SM), and 27% cholesterol. All lipids were synthetic except for PIP, and were obtained from Avanti Polar Lipids (Alabaster, Ala.). For membranes labeled with 1,2-dioleoylphosphatidylethanolamine-N-(1-pyrenesulfonyl) (Py-PE), 2% of PLPE was replaced with Py-PE (Molecular Probes, Eugene, Oreg.). The protein solution was incubated at C in a 4×4 mm2 rectangular quartz cuvette, and titrated with increasing concentrations of either the unlabeled vesicles or vesicles labeled with 2% Py-PE. After each addition of lipid, fluorescence emission spectra were recorded between 300 and 480 nm, using excitation and emission slit bandwidths of 2 and 10 nm, respectively, with an excitation wavelength of 290 nm. Upon binding of the protein to the vesicle membranes containing Py-PE, tryptophan fluorescence was decreased due to RET from tryptophan to Py-PE. The effect of RET was corrected for changes in fluorescence upon addition of the unlabeled vesicles, and the corrected values of ΔF=F₀−F were plotted against total lipid concentration (F and F₀ are tryptophan fluorescence intensities at 340 nm in the presence of a certain lipid concentration and without lipoid, respectively). The experimental binding data, i.e. the dependence of ΔF on the lipid concentration, [L], were fitted with theoretical binding isotherms (Qin et al., 2004):

$$Δ F = Δ {F}_{max} ( \frac{b}{2} - \sqrt{\frac{{b}^{2}}{4} - \frac{δ [ L ]}{N [ P ]}} ) \\ where \\ b ≡ 1 + \frac{δ [ L ]}{N [ P ]} + \frac{{K}_{D}}{[ P ]} \qquad ( 6 )$$

Here, N is the number of lipid molecules in the outer leaflet of membranes per bound protein, [P] is the protein concentration, K_{D} is the dissociation constant, and δ is the fraction of lipid that is accessible to the protein, i.e. the fraction of total lipid in the external leaflet of vesicle membranes (δ=0.52 for 100 nm vesicles, see Qin et al., 2004).

Identification of CDP138 as a novel Akt2 substrate using SILAC-based quantitative proteomics. Quantitative proteomics using a mass spectrometry (MS)-based approach termed stable isotope labeling with amino acids in cell culture (SILAC), have provided a highly sensitive tool to identify and quantify phosphorylated proteins in cultured cells (Kruger et al., 2008; Olsen et al., 2006). To identify potential new Akt substrates in insulin-stimulated adipocytes, three parallel cultures of 3T3-L1 preadipocytes were metabolically labeled with different SILAC amino acids to make their proteomes distinguishable, before differentiated adipocytes were treated with or without the PI3K inhibitor wortmannin followed by insulin as outlined in FIG. 1A. Equal amounts of total cell lysate from the three different samples were pooled together before subjecting to immunoprecipitation to enrich for phosphorylated Akt substrates (PAS) using antibodies (Ab) against the PAS motif RXRXXS/T (Alessi et al., 1996; Obata et al., 2000). The tryptic peptides were subsequently analyzed by tandem mass spectrometry (MS). Among the phosphoproteomic hits was a previously uncharacterized 138-kDa C2 domain-containing phosphoprotein (CDP138), encoded by 5730419I09Rik (kiaa0528). CDP138 was enriched 2.8-fold by insulin and this was inhibited by wortmannin (FIG. 1A). CDP138 contains a predicted C2 domain similar to the Ca²⁺ receptor synaptotagmins, known to be required for vesicle exocytosis (Bai and Chapman, 2004).

We constructed a cDNA clone (IMAGE: 40125694, GI: 109658767) encoding the full-length human kiaa0528 protein tagged with three N-terminal HA epitopes. As shown in FIG. 1B (left panel), insulin stimulates phosphorylation of HA-tagged CDP138 in CHOT cells, as detected with PAS antibodies. Insulin-stimulated phosphorylation was significantly inhibited by wortmannin. An antibody to a peptide from CDP138 was used to analyze endogenous protein in 3T3-L1 adipocytes by immunoprecipitation and immunoblotting (FIG. 1B right panel). CDP138 from insulin-treated cells migrated slower in SDS-PAGE than from control cells and the apparent size shift was reversed by LY294002, a PI3K inhibitor. This pattern of migration is consistent with CDP138 being phosphorylated in insulin-stimulated cells. We detected multiple phosphorylation sites in CDP138 by mass spectrometric measurements (FIG. 1A, lower panel). To determine if Akt2 can directly phosphorylate CDP138, HA-CDP138 was expressed in HEK293T cells and immunoprecipitated with anti-HA Ab before being subjected to in vitro kinase assay in the presence of constitutively active myristoylated Akt2 (myr-HA-Akt2) and γ-³²P-ATP. FIG. 1C shows that active Akt2 does induce CDP138 phosphorylation, demonstrating that CDP138 is an Akt2 substrate. MS analysis of an HA-CDP138 sample revealed that active Akt2 induces CDP138 phosphorylation at serine (Ser) 197, which lies within a consensus Akt substrate motif RQRLIS197 (FIG. 1C). CDP138 protein is highly expressed in insulin-sensitive tissues such as liver, muscle, and fat (FIG. 1D, left panel). Interestingly, CDP138 present in heart and skeletal muscle tissue showed a similar gel shift on SDS-PAGE to that observed in insulin-stimulated cell lysates, suggesting that CDP138 might be phosphorylated or different isoforms are presented in those tissues. As shown in FIG. 1D (middle & right panels), the CDP138 protein level, similar to that of IRS1, is significantly reduced in fat tissue from insulin resistant ob/ob mice, suggesting that CDP138 is a highly regulated protein in insulin sensitive tissues.

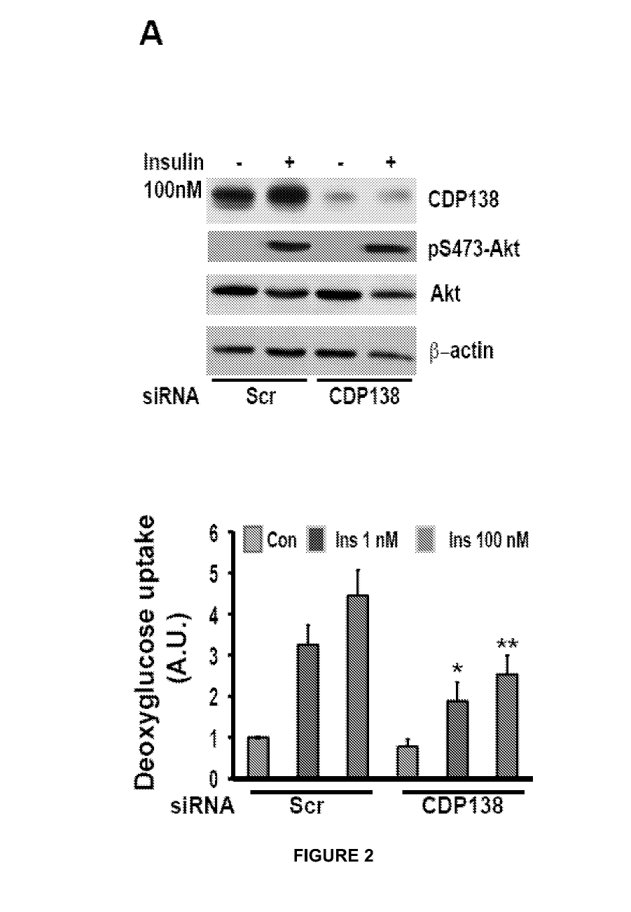

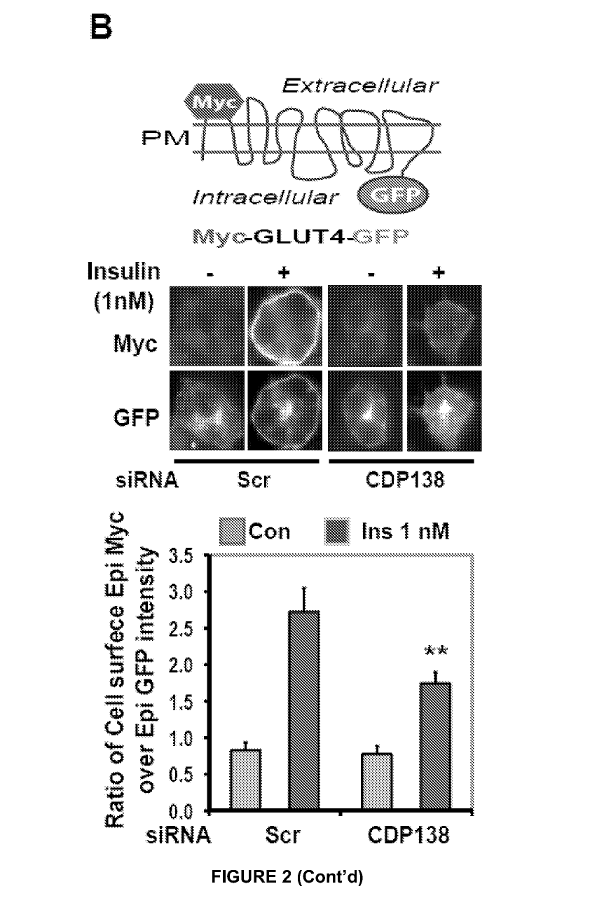

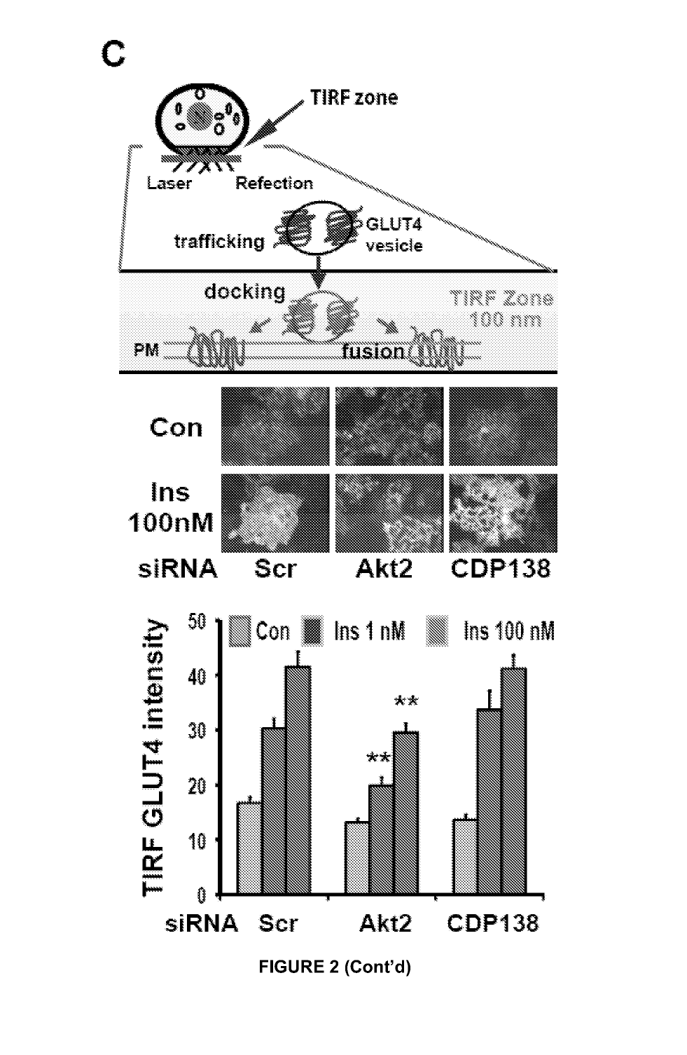

CDP138 is required for insulin-stimulated glucose transport and GLUT4 translocation. Since activation of the Akt2 pathway is important for insulin-stimulated glucose transport and C2 domain-containing proteins such as synaptotagmins are known to be involved in membrane trafficking, we next determined whether loss of CDP138 affects insulin-stimulated glucose transport in adipocytes. As shown in FIG. 2A (upper panel), siRNA-induced knockdown of CDP138 in 3T3-L1 adipocytes reduced protein levels by about 80% without significant effects on insulin-induced Akt phosphorylation or other protein expression, as compared with cells transfected with scrambled siRNA. The reduction in CDP138 protein levels was accompanied by a decrease in insulin-induced glucose transport by about 40% (FIG. 2A lower panel), suggesting that CDP138 is required for glucose transport. To determine whether the reduced glucose transport was due to an effect on the GLUT4 exocytic pathway, we performed GLUT4 translocation assays in 3T3-L1 adipocytes transfected with CDP138 siRNA or the scrambled siRNA, together with the DNA construct encoding a myc-GLUT4-GFP fusion protein with a myc epitope inserted in the first exofacial loop and GFP at the C-terminus of GLUT4 (FIG. 2B) (Jiang et al., 2002). GLUT4 translocation is quantified by measuring the ratio of cell surface myc signal detected by anti-myc immunofluorescence staining, to the total GFP intensity as the myc-GLUT4-GFP expression level in non-permeablized cells. At low concentrations (1 nM), insulin stimulated a 3-fold increase in GLUT4 translocation (FIG. 2B), and CDP138 gene-specific silencing resulted in a 43% decrease of insulin-stimulated GLUT4 translocation, suggesting that CDP138 is critical for the GLUT4 exocytic pathway.

CDP138 is not required for endogenous GLUT4 accumulation at the periphery of the adipocytes. We also quantified endogenous GLUT4 redistribution to the PM using total internal reflection fluorescence microscopy (TIRFM) with a setting of about 100 nm distance from the coverslip (FIG. 2C, top panel). For this experiment, endogenous GLUT4 was detected by immunofluorescent staining with a goat anti-GLUT4 Ab that recognizes the cytoplasmic C-terminus of GLUT4. The fluorescent signal therefore reflects the presence of GLUT4 in the TIRF zone, either inserted in the PM and/or GSV docked at the PM. FIG. 2C shows the TIRFM images and quantification of GLUT4 distribution in the TIRF zone. In 3T3-L1 adipocytes transfected with scrambled siRNA, 100 nM insulin enhanced the GLUT4 signal in the TIRF zone by about 2.5-fold. Cells transfected with Akt2 siRNA showed a significant reduction in insulin-stimulated GLUT4 distribution to the periphery, consistent with the concept that Akt2 plays a role in GLUT4 trafficking. In contrast, knockdown of CDP138 did not inhibit insulin-stimulated GLUT4 accumulation at the periphery (FIG. 2C). This finding appears inconsistent with the results obtained with the myc-GLUT4-GFP translocation assay that showed reduction of GLUT4 on the cell surface by silencing CDP138 (FIG. 2B). However, both observations are consistent with the possibility that CDP138 is specifically required for the insertion of GLUT4 into the PM, but not GSV movement to the periphery, whereas Akt2 is known to be involved in both steps.

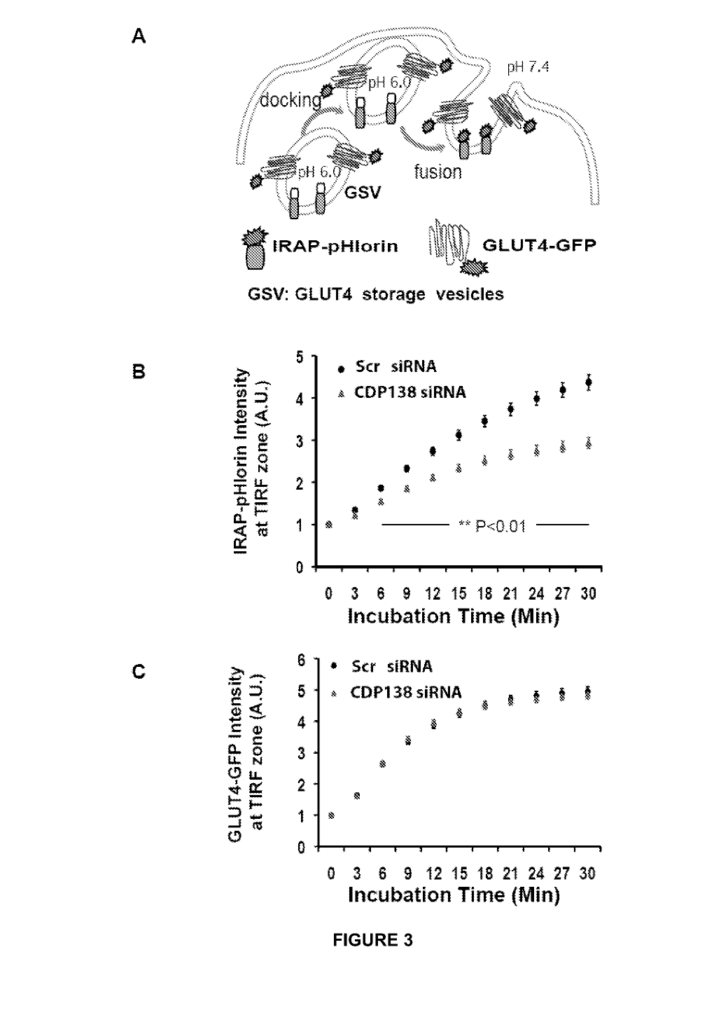

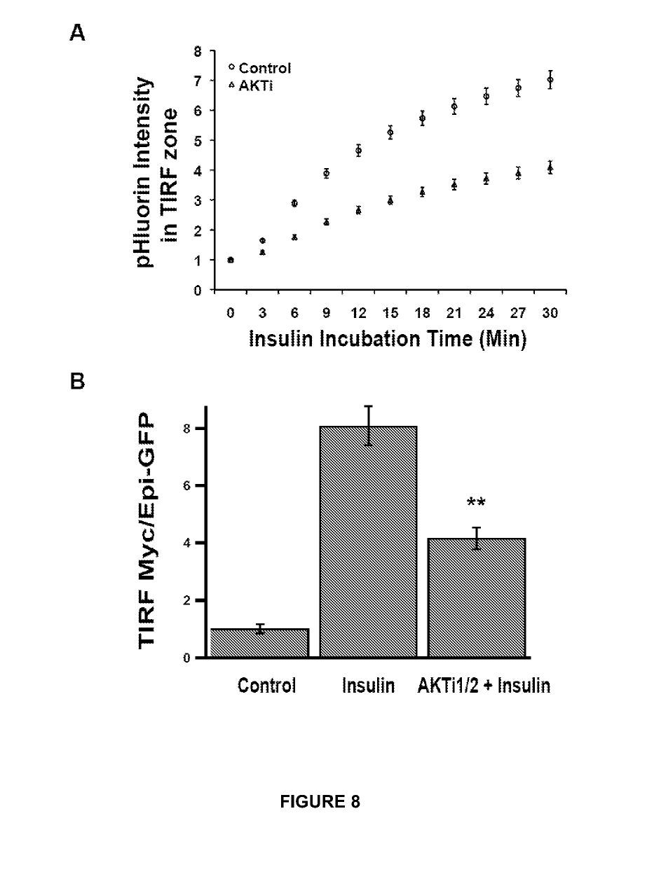

CDP138 is a key factor involved in the process of fusion between GSV and the PM. To determine whether CDP138 is required for insulin-stimulated fusion between GLUT4 vesicles and the PM, we used a TIRFM-based live cell fusion assay (Jiang et al., 2008). The assay is based on the expression of a fusion protein of insulin-responsive aminopeptidase (IRAP) tagged with the pH-sensitive green fluorescence protein pHluorin at its luminal terminus. The resultant molecular probe (IRAP-pHluorin) co-localizes with the insulin-responsive GSV in 3T3-L1 adipocytes. IRAP-pHluorin is essentially non-fluorescent at pH 6.0 within the GSV, but is very brightly fluorescent at the pH 7.4 environment when GSV have fused with the PM as illustrated in FIG. 3A (Jiang et al., 2008; Lopez et al., 2009). Therefore, this molecular probe provides a sensitive tool to monitor dynamic changes in GSV-PM fusion, with the pHluorin fluorescence intensity reflecting the fusion event. Insulin significantly stimulates GSV fusion with the PM in the TIRF zone in live adipocytes. As reported by Lopez et al (2009), the Akt1/2 inhibitor Akti significantly inhibited both insulin-induced membrane fusion in live adipocyes and myc-GLUT4-GFP translocation to cell surface in fixed cells (FIG. 8). In comparison to scrambled siRNA-treated cells, knockdown of CDP138 significantly diminished the pHluorin intensity within the first 6 min of insulin treatment, and this decrease was sustained over a 30 min period (FIG. 3B). Quantitatively, knockdown of CDP138 inhibited the insulin-induced IRAP-pHluorin signal by about 35% (FIG. 3B). We also determined if CDP138 is required for insulin-stimulated GLUT4-EGFP accumulation in the TIRF zone in live adipocytes. Knockdown of CDP138 did not significantly affect GLUT4-EGFP accumulation in the TIRF zone (FIG. 3C). Taken together, our data suggests that CDP138 is specifically required for insulin-stimulated GSV fusion with the PM, but not for the movement of the vesicles from intracellular stores to the TIRF zone.

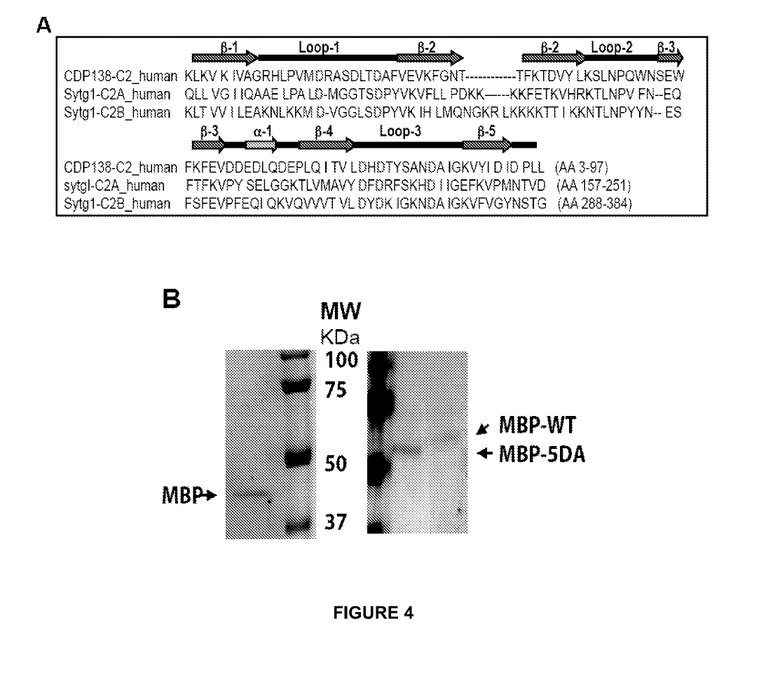

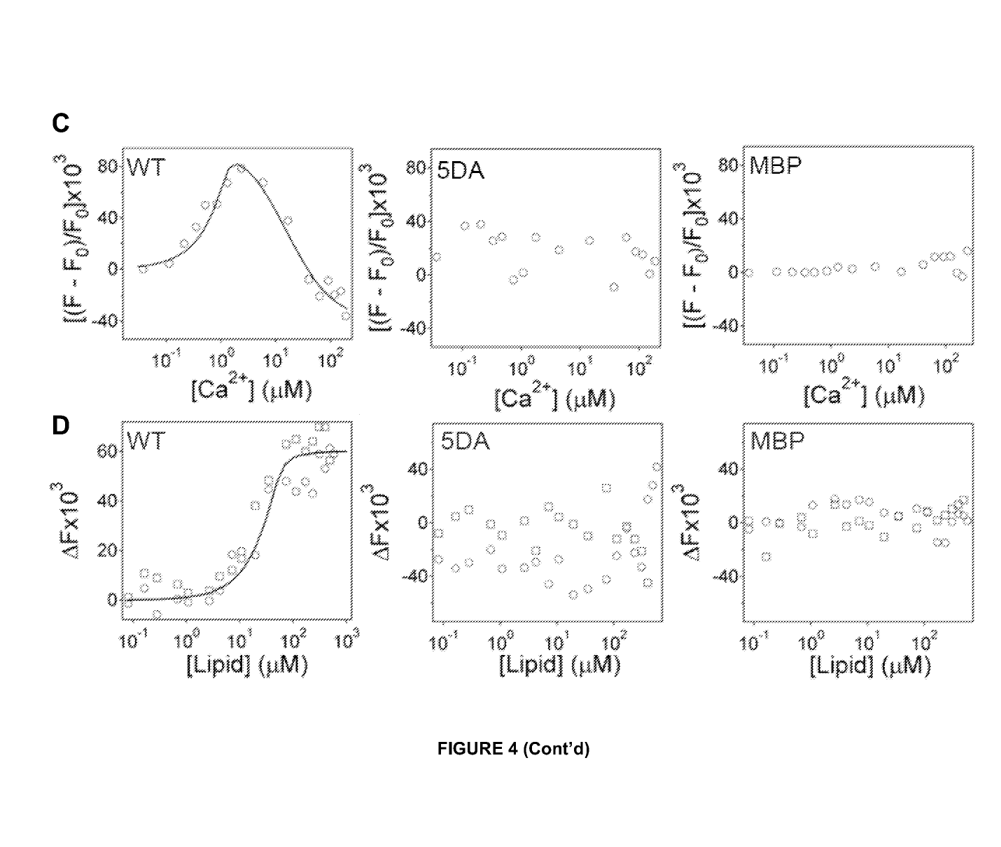

The C2 domain of CDP138 has two Ca²⁺-binding sites that are critical for membrane lipid binding. The primary amino acid sequence of the CDP138 C2 domain is similar to the C2A and C2B domains from synaptotagamin-1, with 5 conserved aspartate residues in loop 1 and loop 3 regions (FIG. 4A) that may interact with Ca²⁺. To test this possibility, we performed biophysical and functional analyses on the isolated C2 domain prepared in E. coli. The CDP138 C2 domain fused to maltose-binding protein (MBP) is soluble. Purified MBP, MBP-C2-WT and MBP-C2-5DA, a mutant lacking five aspartate residues (FIG. 4B), have been analyzed for their Ca²⁺ and lipid membrane binding properties. The mutant C2-5DA domain fusion protein migrates faster in SDS-PAGE than the wild type fusion protein, because the substitution of 5 aspartate residues with alanine changes the molecular weight and charge of the protein.

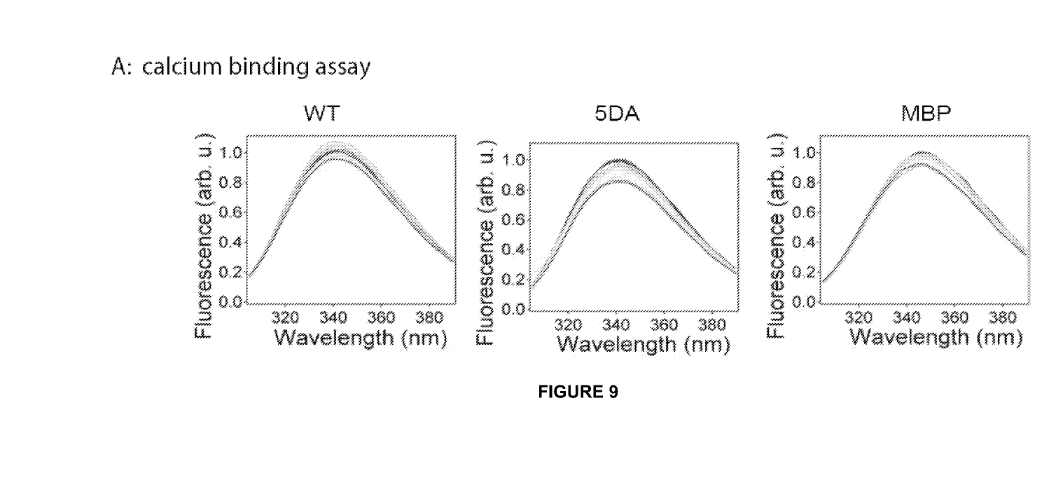

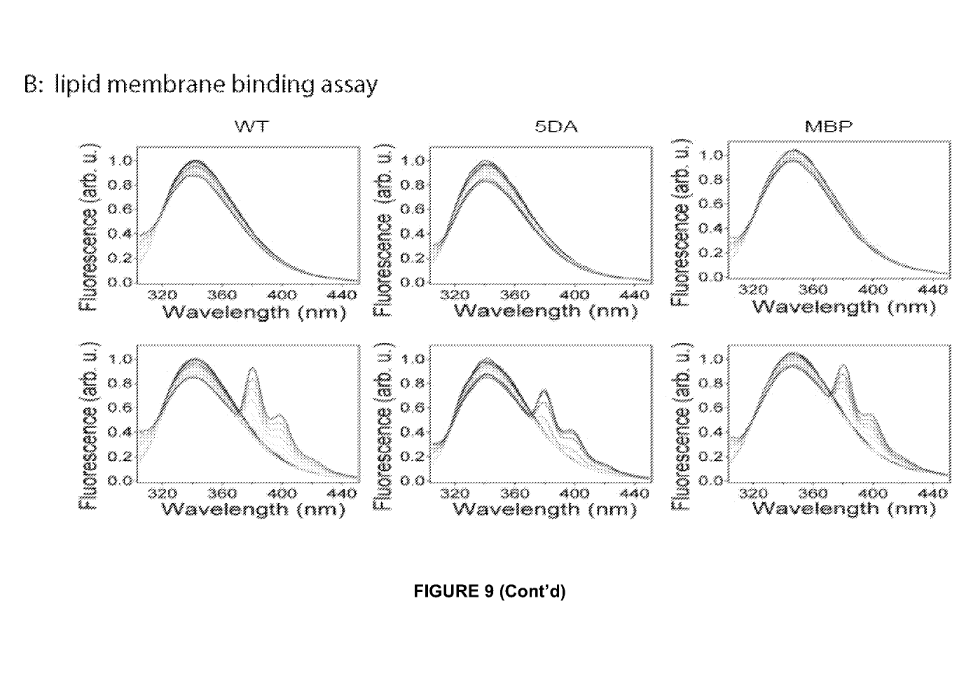

Ca²⁺-binding property of the C2 domain. Calcium binding to the fusion proteins was directly measured by assessing Ca²⁺-induced changes in tryptophan fluorescence (FIG. 9A). The data in FIG. 4C show that increasing Ca²⁺ concentrations exert a biphasic effect on the fluorescence of the wild type protein; fluorescence intensity increases at Ca²⁺ concentrations up to 2-3 μM and then decreases at higher Ca²⁺ concentrations. Analysis of the biphasic effect indicated that the wild-type protein has two Ca²⁺-binding sites; a high affinity binding site with KD=0.03 μM, and a lower affinity site with KD=15.0 μM. The effect of Ca²⁺ ions on the fluorescence of the 5DA mutant protein and on MBP was negligible (FIG. 4C).

Binding of C2 domain to lipid membranes. Protein-lipid membrane interactions were studied by resonance energy transfer (RET), as described earlier (Qin et al., 2004). The fusion proteins were incubated with lipid vesicles containing 2% Py-phosphatidylethanolamine (Py-PE) and tryptophans were excited at 290 nm. If the protein binds to the membrane, the energy of the excited electrons of tryptophan's indole ring can be transferred to the pyrene group of Py-PE. This results in a decrease in tryptophan fluorescence emission at around 340 nm and generates pyrene fluorescence peaks between 370 and 430 nm, which indicates protein binding to the membranes. The data shown in FIG. 4D demonstrate clearly that the RET effect is only observed with the wild type C2 domain, but not the 5DA mutant or MBP proteins. The spectra sets collected when the proteins were titrated with unlabeled and Py-PE-labeled vesicles are presented in FIG. 9B. These data were used to determine lipid concentration-dependence of the relative change in tryptophan fluorescence intensity at 340 nm, ΔF₃₄₀, corrected for the effect of unlabeled vesicles (FIG. 4D). For the wild type C2 domain the data predict lipid-to-protein stoichiometry of N=20 and a dissociation constant of KD=0.06 μM. Data for the 5DA and MBP proteins did not indicate membrane binding.

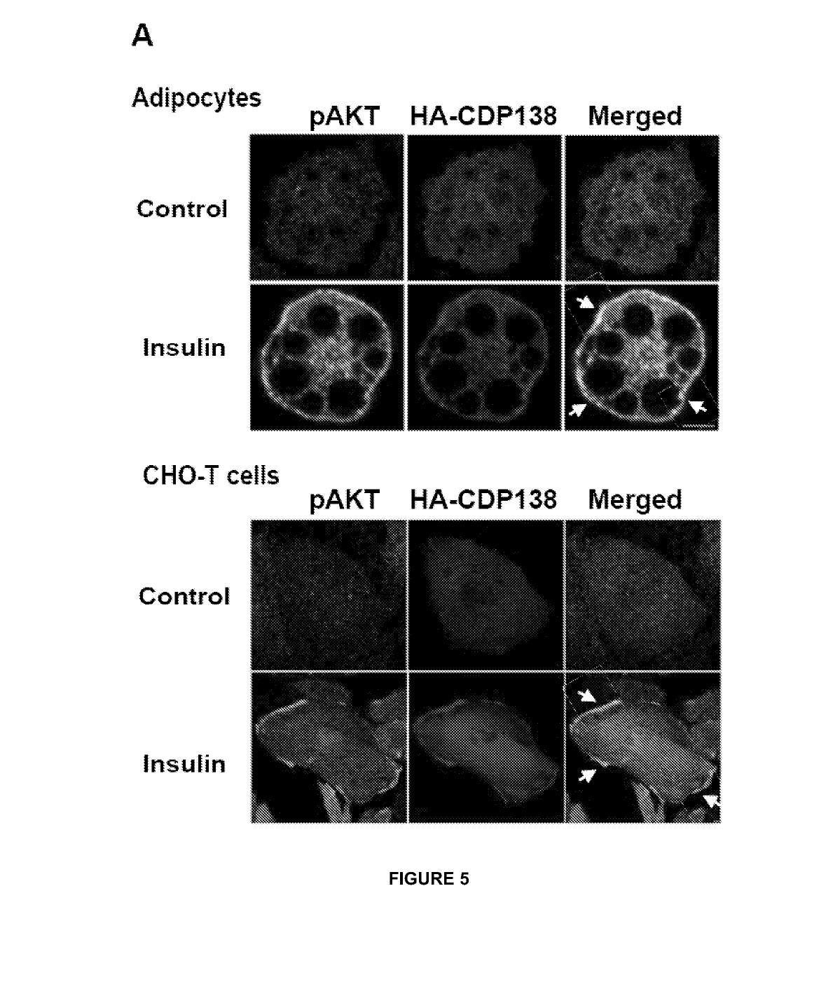

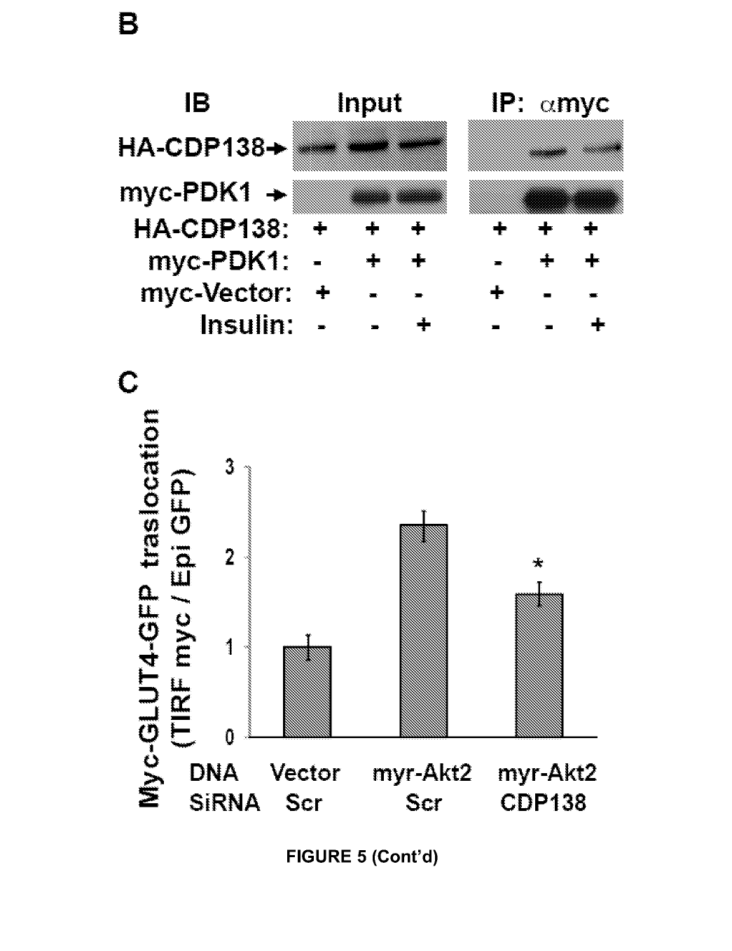

CDP138 is partially co-localized with active Akt and is required for constitutively active Akt2-induced GLUT4 translocation. To examine if CDP138 co-localizes with Akt, adipocytes and CHOT cells were transfected with HA-CDP138 expression vector. We consistently observed that insulin stimulated HA-CDP138 co-localization with active Akt, detectable with phospho-S473 Akt specific antibody, at the PM cortical area in adipocytes, and at the membrane ruffles in CHOT cells (FIGS. 5A & B).

It is established that constitutively active Akt induces GLUT4 translocation independently of insulin stimulation. To determine if CDP138 functions downstream of Akt2, differentiated adipocytes were transfected with active myr-HA-Akt2 and myc-GLUT4-GFP together with either the scrambled siRNA, or siRNA against mouse CDP138. TIRFM was then used to quantify the effect of active myr-HA-Akt2 on the translocation of myc-GLUT4-GFP to the cell surface. As shown in FIG. 5C, overexpression of active Akt2 stimulated GLUT4 translocation by about 2.5-fold in adipocytes transfected with the scrambled siRNA. However, siRNA-induced knockdown of CDP138 significantly blocked the effect of constitutively active Akt2 on GLUT4 translocation. As noted above, knockdown of CDP138 did not alter insulin-stimulated Akt phosphorylation (FIG. 2A). Together, these data confirm that CDP138 functions downstream of the Akt2 pathway and is required for Akt2-induced GLUT4 translocation.

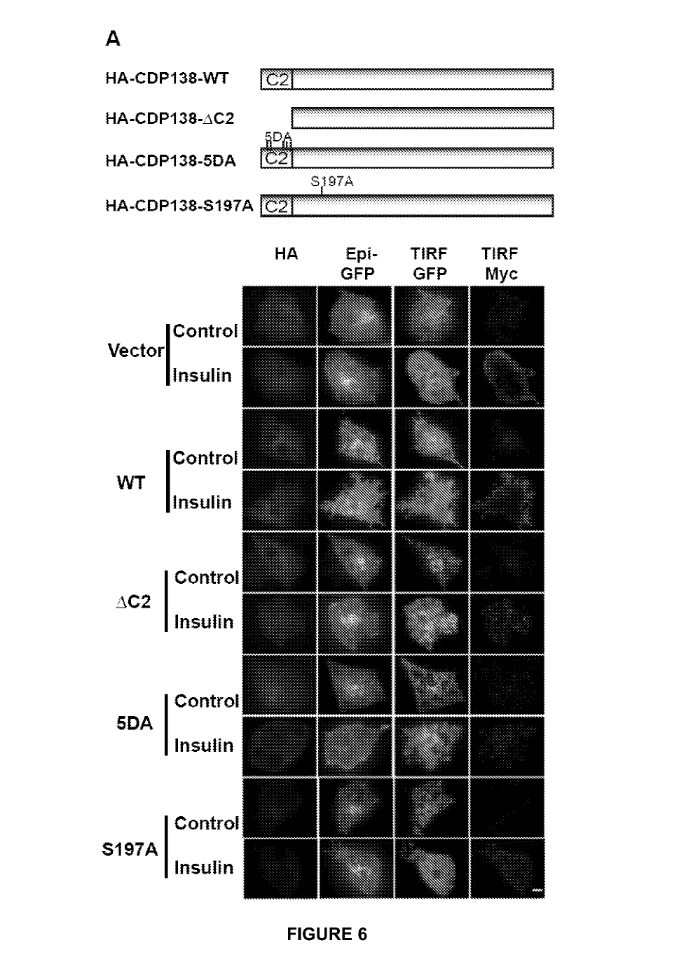

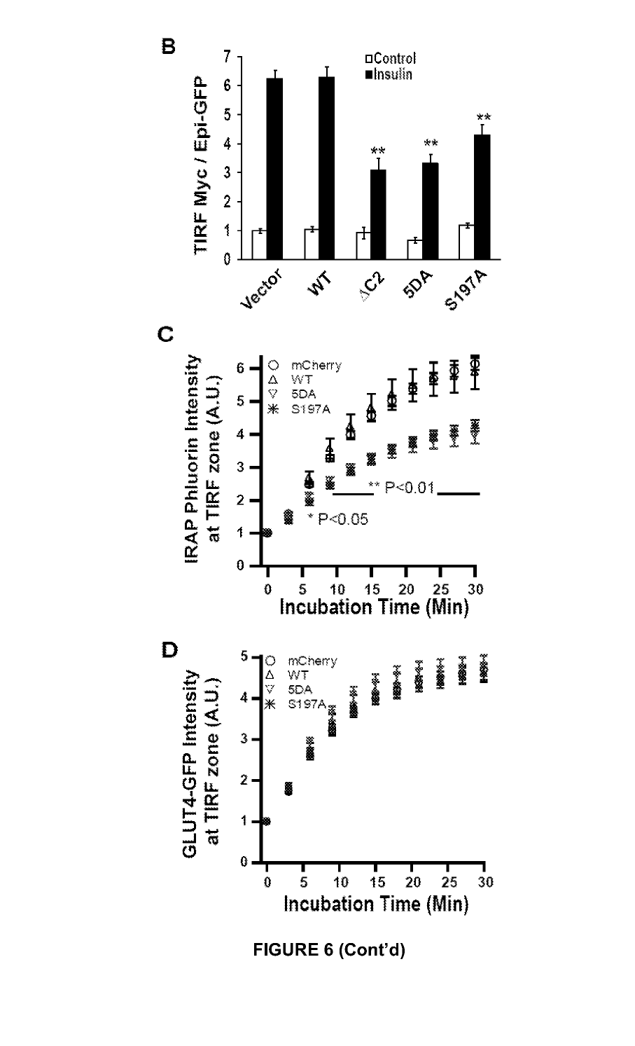

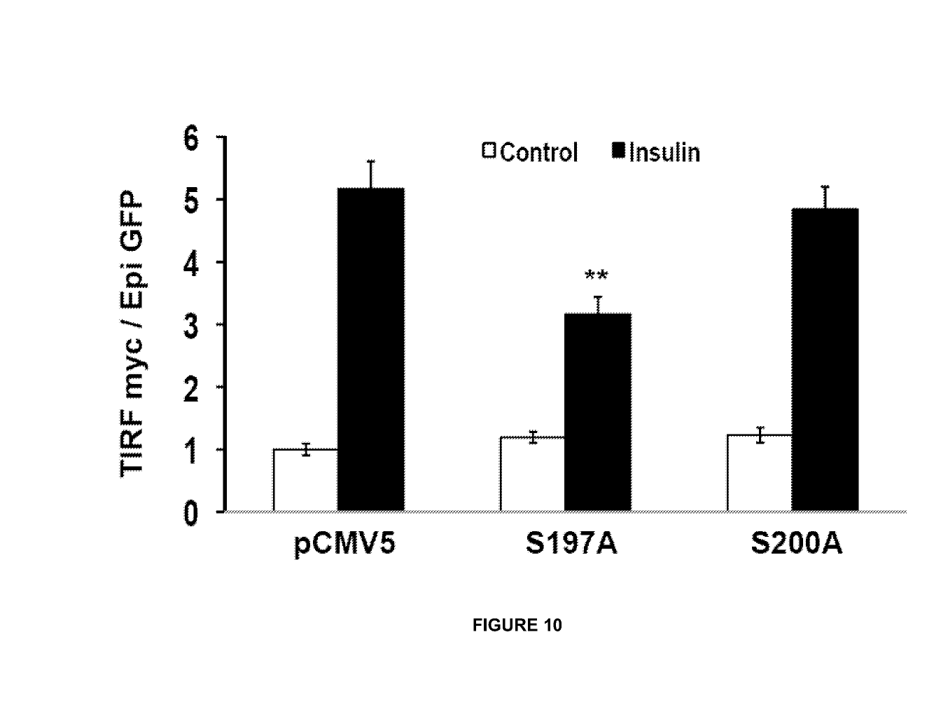

The C2 domain and Akt phosphorylation site are both required for the normal function of CDP138 in GLUT4 translocation. We next determined if the C2 domain and Akt2 phosphorylation site in CDP138 are necessary for GLUT4 translocation. Myc-GLUT4-GFP translocation was tested in the presence of the HA-CDP138-WT or mutant proteins that lack (a) the C2 domain (ΔC2 AA1-108), (b) the Ca²⁺-binding aspartate residues (5DA), or (c) the Akt2 phosphorylation site Ser197 (S197A). Insulin-stimulated Myc-GLUT4-GFP translocation was quantified as the ratio of cell surface myc signal (detected with TIRFM) to the total GFP signal in the widefield image (Epi-GFP). As shown in FIGS. 6A and 6B, overexpression of HA-CDP138-WT did not significantly alter GLUT4 translocation. However, overexpression of all three constructs (HA-CDP138-ΔC2, HA-CDP138-5DA, and HA-CDP138-S197A) inhibited the insulin-stimulated translocation of myc-GLUT4-GFP to the cell surface, with the ΔC2 construct showing the most inhibitory effects. Our phosphopeptide analysis showed that CDP138 is also phosphorylated at the Ser200 residue. Thus, we compared the effects of overexpressed mutants lacking either Ser197 or Ser200 on insulin-stimulated myc-GLUT4-GFP translocation. Interestingly, only S197A, but not S200A, blocked GLUT4 translocation (FIG. 10), further suggesting that Akt2-dependent phosphorylation of Ser197 is necessary for CDP138 function but phosphorylation of Ser200 is not.

We have also constructed similar mutants of CDP138 as described above but with mCherry fused at their C-terminus, and compared their effect on insulin-stimulated GLUT4 trafficking and GSV-PM fusion, as detected with TIRFM in live adipocytes using GLUT4-EGFP and IRAP-pHluorin as the molecular probes, respectively. Our data show that the CDP138-5DA-mCherry and CDP138-S197A-mCherry mutants inhibited membrane fusion, but the CDP138-WT-mCherry or mCherry control vector had no effect (FIG. 6C). Despite their effect on membrane fusion, none of the constructs significantly affected the insulin-stimulated accumulation of GLUT4-EGFP in the TIRF zone (FIG. 6D). These data suggest that Akt2-induced phosphorylation and Ca²⁺-binding by CDP138 are both important for GSV-PM fusion, but not GSV trafficking in adipocytes.

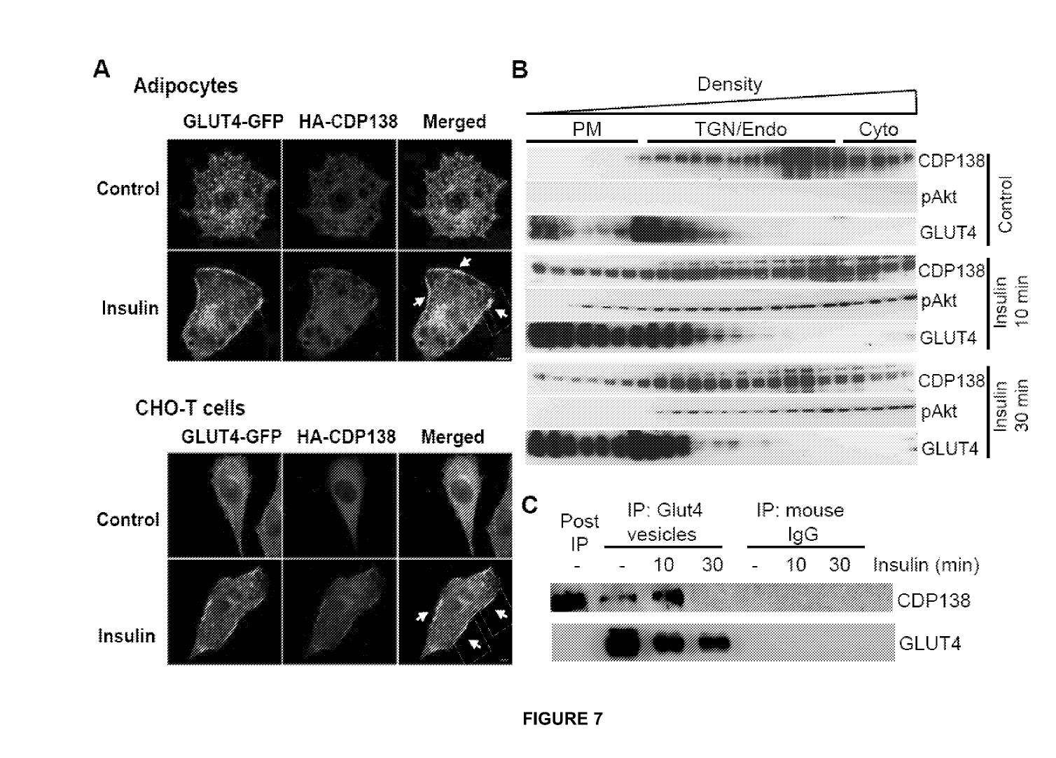

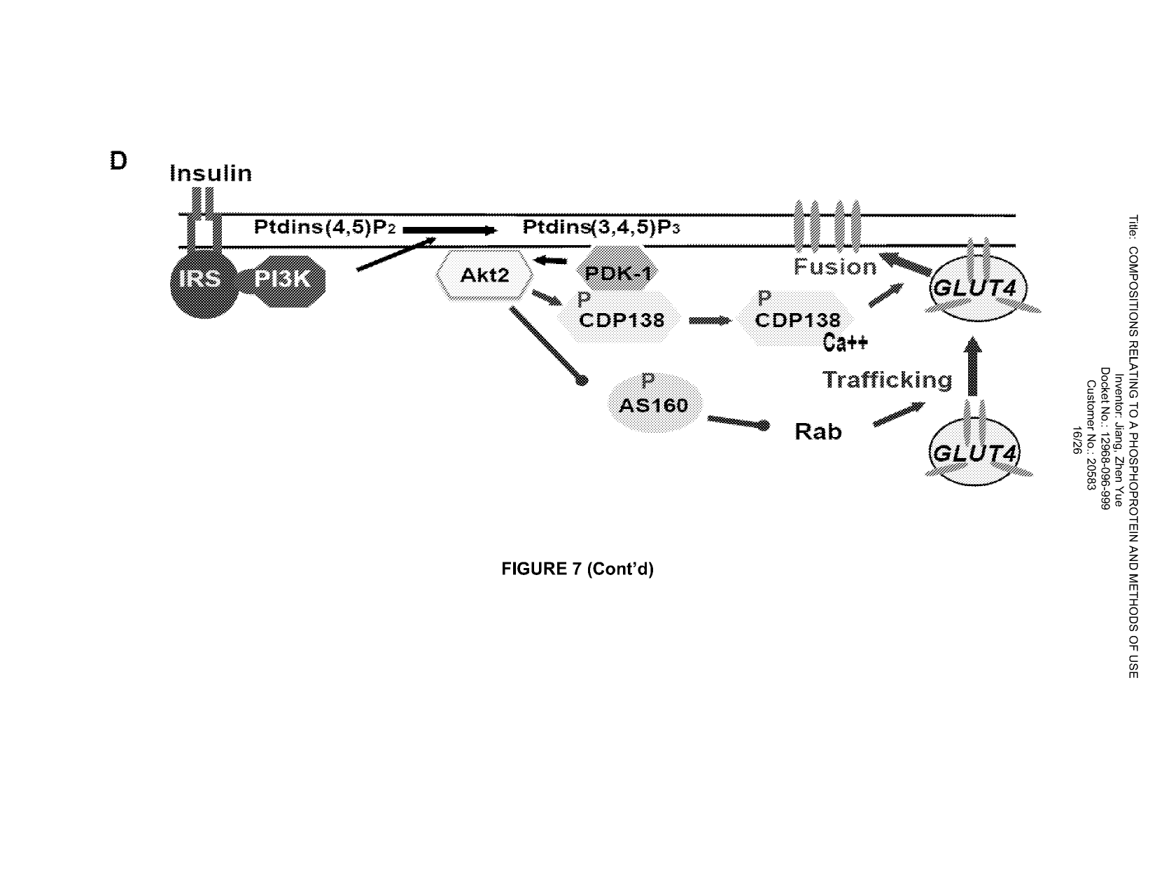

CDP138 is dynamically associated with the PM and GLUT4 vesicles. To examine the intracellular distribution of CDP138, we co-expressed myc-GLUT4-GFP and HA-CDP138 in adipocytes. As shown in FIG. 7A, in the basal state intracellular staining of HA-CDP138-WT was punctuate and only partially overlapped with GLUT4 vesicles in intracellular stores. Within 10 minutes of insulin stimulation, GLUT4 and CDP138 can be seen co-localized at the PM. A similar pattern was observed in stable CHO-T cell lines expressing myc-GLUT4-GFP (FIG. 7A). Next, we used self-generated iodixanol gradient fractionation to examine the subcellular distribution of CDP138 and GLUT4 in adipocytes. CDP138 was partially co-fractionated with GLUT4 in the medium density fractions at the basal state and redistributed to the lower density PM fractions within 10 min of insulin stimulation (FIG. 7B). After 30 minutes, CDP138 like phospho-Akt had partially dissociated from the PM. GLUT4 accumulated in PM fractions within 30 min of insulin stimulation. We also analyzed CDP138 distribution in GLUT4 vesicles enriched with conjugated monoclonal anti-GLUT4 Ab (1F8). Interestingly, insulin also stimulated the association of CDP138 with GLUT4 vesicles within 10 min (FIG. 7C). However, this association was undetectable 30 min after stimulation when total GLUT4 content in the enriched vesicles was also reduced by about 35% (FIG. 7C), possibly due to fusion between GLUT4 vesicles and the PM. These data suggest that PPC2D dynamically interacts with both the PM and GLUT4 vesicles.

It is known that activation of Akt2 is required for insulin-stimulated GLUT4 translocation, and that Akt2 acts by regulating mobilization of GSV and fusion between GSV and the PM (Zaid et al., 2008). It has been reported previously that Akt2 controls GLUT4 retention and trafficking by phosphorylating the RabGAP AS160. However, the molecular mechanism by which Akt2 regulates GLUT4 insertion into the PM, a rate-limiting step of GLUT4 translocation, remains unclear. In the present study, we used a SILAC quantitative phosphoproteomic approach to identify CDP138, a previously unknown C2 domain-containing phosphoprotein, and confirmed that it is an Akt2 substrate. RNAi-based functional assays revealed that CDP138 is required for insulin-stimulated glucose transport and GLUT4 translocation to the PM, but not for GLUT4 movement to the periphery in adipocytes. We used both pH-sensitive IRAP-pHluorin and GLUT4-EGFP as molecular probes to demonstrate in live adipocytes that CDP138 is critical for membrane fusion between GSV and the PM, but not for GSV trafficking to the TIRF zone. Collectively, these complementary functional analyses demonstrate that the novel phosphoprotein CDP138 is involved in regulating GLUT4 translocation, most likely at the GSV-PM fusion step. Thus, CDP138 represents a novel link between Akt2 activation and GLUT4 insertion into the PM. It is possible that Akt2 regulates the GLUT4 trafficking and membrane fusion steps in adipocytes through the RabGAP AS160 and CDP138, respectively (FIG. 7D).

Our results are consistent with the hypothesis that CDP138 is a downstream target of Akt2 and is involved in the regulation of GLUT4 translocation. First, insulin stimulated phosphorylation of CDP138 in cultured cells, and the phosphorylation was partially blocked by the PI3K inhibitors. We also detected several phosphorylation sites in CDP138 by mass spectrometry, which suggests that insulin might induce CDP138 phosphorylation at different sites through both PI3K-dependent and -independent pathways. Second, we showed that constitutively active Akt2 induced CDP138 phosphorylation at a Ser197 residue within a consensus Akt substrate motif. Over-expression of a mutant CDP138 that was lacking the Ser197 phosphorylation site, but not Ser200, significantly blocked insulin-stimulated myc-GLUT4-GFP translocation to the cell surface and GSV-PM fusion in adipocytes, suggesting that Ser197 phosphorylation is important to the glucose transporter system. Third, our results showed that CDP138 co-localizes with phospho-Akt in insulin-stimulated cells. We also observed that CDP138 interacts with Akt2 upstream kinase PDK1 in a proteomics study and this was confirmed in a co-immunoprecipitation study (Data not shown). These observations suggest the interesting possibility that the CDP138-PDK1 interaction might bring CDP138 and phospho-Akt2 in close proximity at the PM (FIG. 7D). This might be mediated through PI3K-derived PI(3,4,5)P3, which interacts with the PH domains of both PDK1 and Akt2 in insulin-stimulated cells. If this occurs, CDP138 would become accessible to phosphorylation by active Akt2. Furthermore, RNAi-induced gene specific knockdown of CDP138 did not affect insulin-stimulated Akt phosphorylation but significantly inhibited GLUT4 translocation induced by constitutively active Akt2, suggesting this novel phosphoprotein functions downstream of the Akt2 pathway.

To understand the molecular mechanism by which CDP138 regulates GLUT4 translocation, we also analyzed the biochemical and functional interactions of the C2 domain-containing protein. Deletion of the C2 domain from CDP138 significantly inhibited insulin-stimulated GLUT4 translocation, suggesting the C2 domain is crucial for this process. The C2 domain of CDP138 is similar to those of known membrane fusion proteins such as synaptotagmin. Biophysical analyses revealed that the purified C2 domain of CDP138 is able to bind Ca²⁺ and liposomes with a lipid composition that mimics the cytoplasmic face of plasma membranes. It is interesting to note that the C2 domain contains two Ca²⁺-binding sites of differing affinity, presumably one each in loop 1 and 3. The mutant C2 domain lacking five aspartate residues in loop 1 and 3 regions is unable to bind Ca²⁺ or membrane lipids, suggesting that interaction of the C2 domain with lipids is Ca²⁺-dependent. It is possible that Ca²⁺-binding to the C2 domain results in exposure of nonpolar residues that mediate membrane binding. Alternatively, Ca²⁺ ions may serve as ionic bridges between acidic residues of the protein and negatively charged membranes. Interestingly, in the studies using GLUT4 vesicles and density gradient fractionation of membrane compartments, we also observed that CDP138 associated with GLUT4 vesicles and the lower density PM-containing fractions within 10 min of treatment of adipocytes with insulin. Surprisingly, CDP138 dissociated from both the vesicles and the PM-containing fractions in adipocytes after 30 min. These dynamic interactions suggest that CDP138 may form cluster together with GLUT4 vesicle at the PM before membrane fusion occurs. It is possible that CDP138 contributes to the insulin-regulated membrane fusion process through interaction with both the PM and the GSV. Therefore, CDP138 is unique among proteins known to be involved in constitutive membrane fusion processes. Further studies are needed to understand the molecular basis by which CDP138 regulates membrane fusion.

### Example II

### Overexpression of CDP138 Protects Neuronal Cells and Induces Neurite Outgrowth

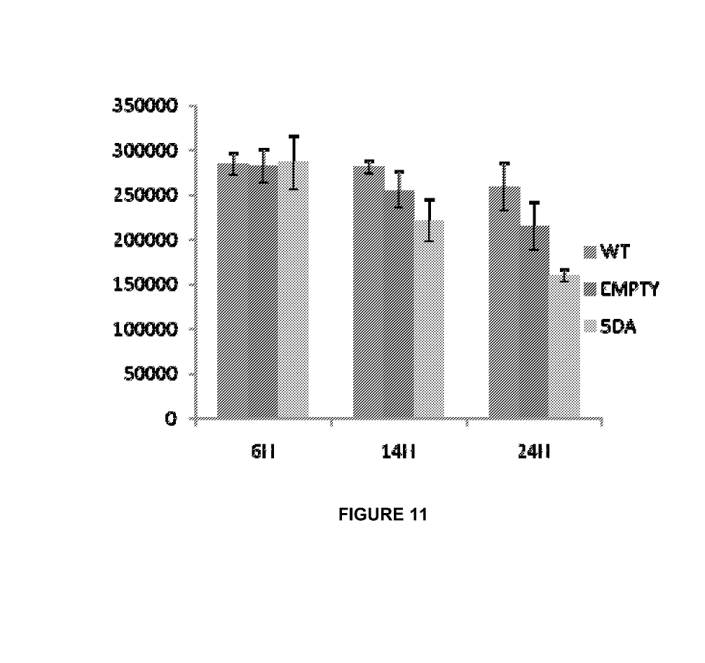

In order to determine the affects of CDP138 of neuronal cell survival, PC12 cells were transfected with wild-type CDP138-WT vector, dominant negative mutant CDP138-5DA vector or an empty vector for 24 h followed by adding NGF (neuron growth factor) for 24 h. Then cells were serum starved for 6 hours, 14 hour, and 24 hour before counting cell number in 2 wells. Within 14 hours of starvation, PC12 cells that were transfected with empty vector or the mutant CDP138-5DA vector showed significant cell death, whereas cells transfected with wild-type CDP138-WT showed no appreciable cell death (FIG. 11). After 24 hours of starvation, the cells transfected with the dominant negative mutant CDP138-5DA vector showed the highest amount of cell death, whereas the cells transfected with the wild-type CDP138 continued to survive (FIG. 11).

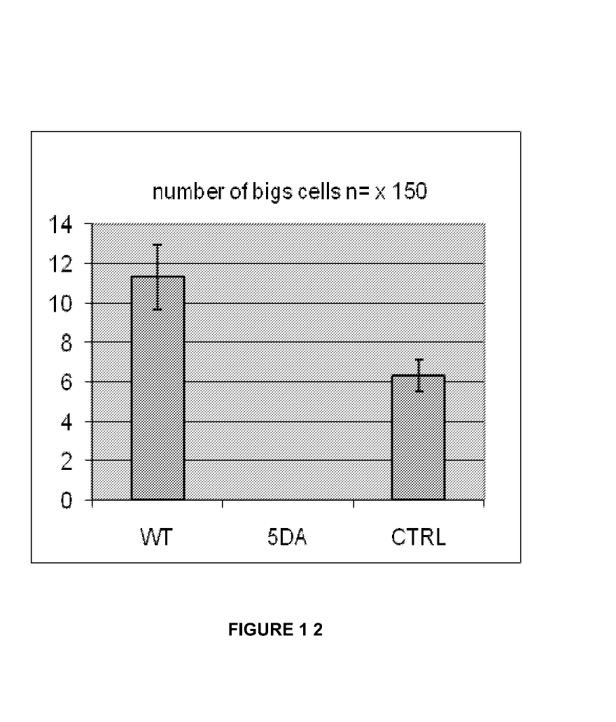

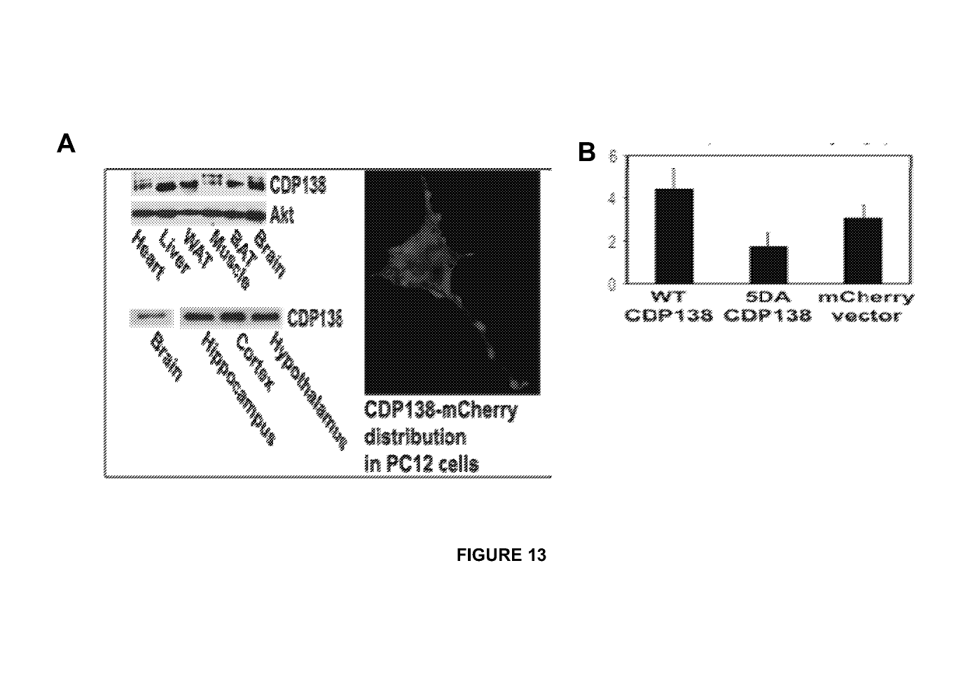

In addition to counting the total number of cells that survived, the number of big surviving differentiated neuronal cells were also assessed following 24 hours of starvation. PC12 cells that were transfected with wild-type CDP138-WT showed approximately 1650 big differentiated neuronal cells per well, while cells that were transfected with an empty vector only showed approximately 900 big differentiated neuronal cells per well (FIG. 12). This was a 50% increase in the number of big differentiated cells. Interestingly, cells that were transfected with the dominant negative mutant CDP138-5DA did not form any big differentiated neuronal cells. Lastly, overexpression of CDP138 in PC12 cells showed enhanced outgrowth of neuritis upon contacting with NGF. FIG. 13B shows the fold over cell body length of PC12 cells that were transfected with a wild-type CDP138-WT vector, mutant CDP138-5DA vector or empty mCherry vector. Overexpressed CDP138-WT showed an approximate 4 fold increase in cell body length, whereas CDP138-5DA mutant showed only approximately 1 fold increase.

These results show that overexpression of wild-type CDP 138 prolongs the survival of differentiated PC12 neuronal cells after serum starvation and enhanced neurite growth while the dominant negative mutant CDP138-5DA shows the opposite effect.

### Example III

### Expression Pattern of CDP138 in Tissues and Neuronal Cells

In order to determine if expression of CDP138 tissue specific, immunoblotting of total protein isolated from heart, liver, white adipose tissue (WAT), muscle, brown adipose tissue (BAT) and brain was conducted. Total protein extracts were separated and assayed using standard Wester blotting techniques. CDP138 showed significant expression in liver, white adipose tissue and brown adipose tissue (FIG. 13A). Interestingly, CDP138 showed the highest expression in brain tissue samples (FIG. 13A). Expression of CDP138 also appeared to be evenly expressed in the hippocampus, cortex and hypothalamus regions of the brain (FIG. 13A). Additionally, when CDP138-mCherry vector was transfected into PC12 cells, expressed CDP138-mCherry protein was found to be equally distributed in the differentiated PC12 cells including being located in the neurite and synapse of cells (FIG. 13A).

### Example IV

### Overexpression of CDP138 Inhibits Cell Proliferation

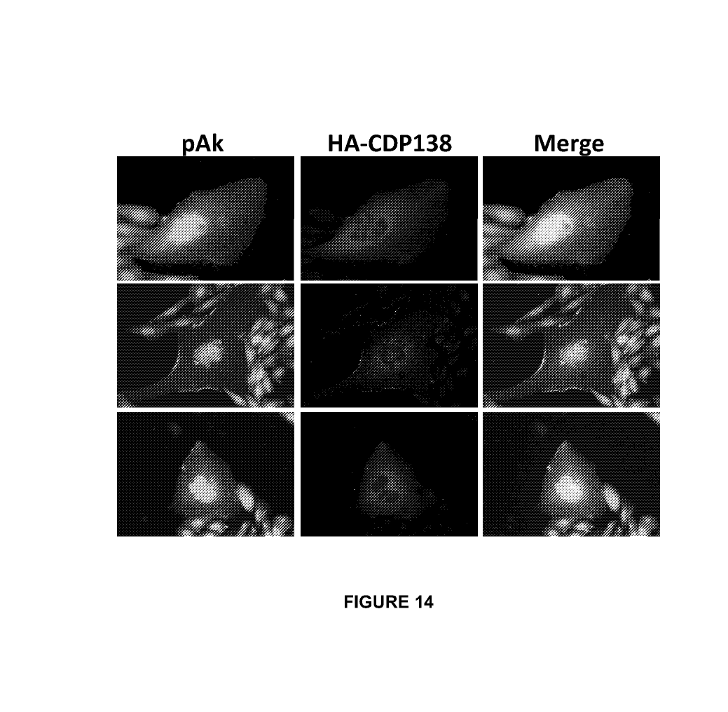

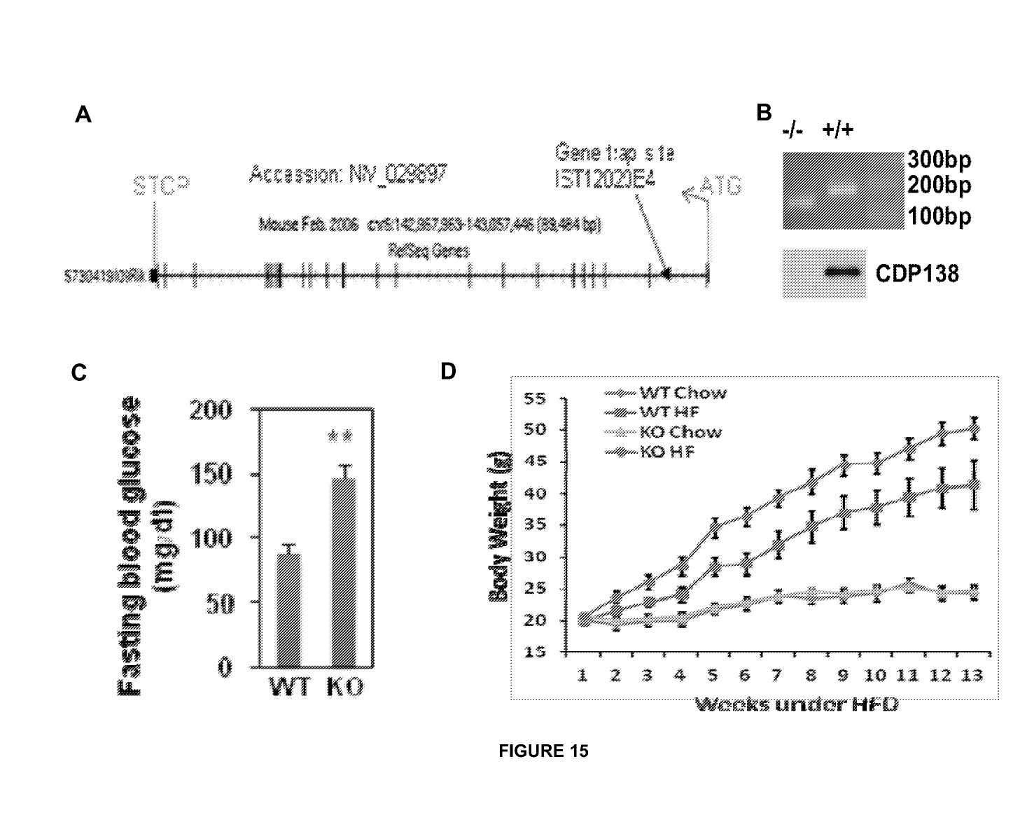

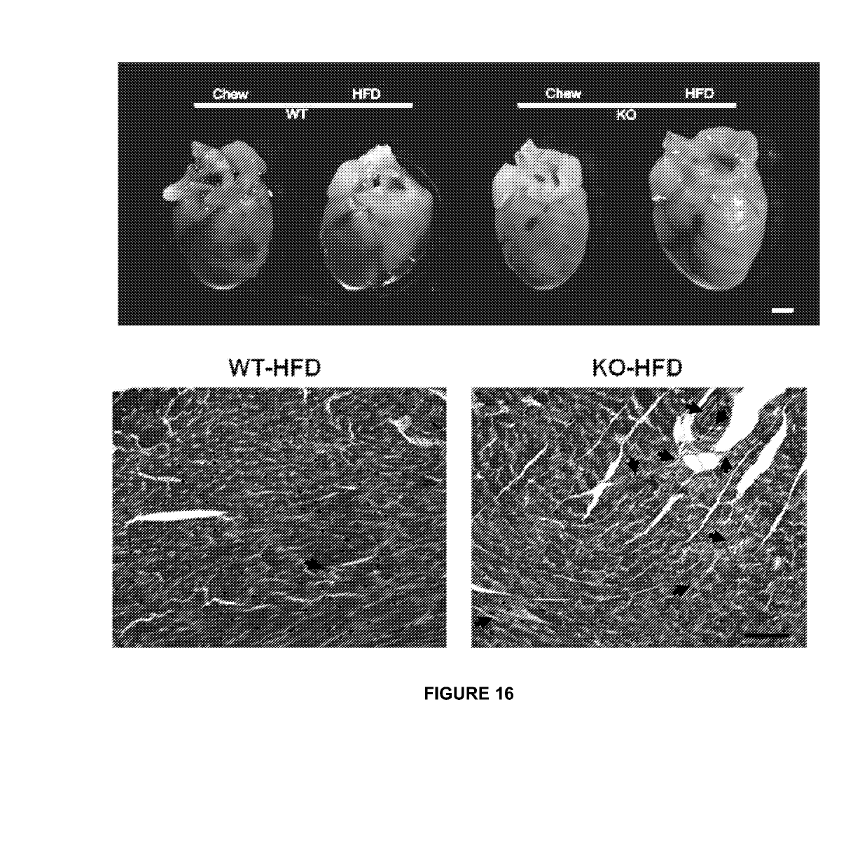

Overexpressed CDP138 inhibits cell division as indicated by large cell body with multiple Nucleus in HA-tagged CDP138 positive CHO-T cells (proliferative cells), suggesting CDP138 can be a tumor suppressor. HA-CDP138 positive cell is 3-5 times bigger than HA-negative cells (FIG. 14). Over the course of three different experiments, about 24.6% of wild-type HA-CDP138 positive cells had big multiple nuclei while the percentage increased to about 51% with a dominant mutant CDP138 positive cells.

### Example V

### Knockout Mice Model

### Knockout Mice of CDP138 are Prone to High-Fat Diet Induced Obesity (Body Weight Gain) and Hyperglycemia

ES cell clones were selected and used for generating CDP138 knockout mice as described in FIG. 1 (A). A mouse gene line lacking CDP138 was generated (FIG. 1B). CDP138 knockout mice and wild type control mice were used for comparing blood glucose levels and body weight gain after challenging with high-fat diet, 60% of calories from fats (Cat# D12492, Research Diet, Inc). This comparison showed that the fasting blood glucose levels in CDP138 null mice (male, n=4) are not significantly different from that in wild-type littermates (male, n=4) at the age of 6-weeks. However, the fasting blood glucose levels in CDP138 null mice are significantly higher than that in WT littermates (C57BL/6N mice) after 4 weeks of feeding with a high fat diet (60 kcal % fat chow) at age of 10 weeks of age (FIG. 1C). This shows that high-fat diet can accelerate the impairment of glucose homeostasis in CDP138 null mice. Interestingly, the CDP138 KO mice are also prone to high fat diet induced body weight gain (FIG. 1D). These results show that CDP138 knockout mice are prone to develop both diabetes and obesity after feeding with western life-style of diet. Therefore, CDP138 can protect mice from high-fat diet-induced diabetes and obesity.

### CDP138 Knockout Mice are Prone to High Fat Diet Induced Cardiac Hyperdrophy and Fibrosis

Surprisingly, the hearts from CDP138 knockout mice fed with 60% high-fat diet (HFD) were shown to be significantly bigger that those from wild type C57BL/6N mice fed with the same diet (FIG. 2 upper panel). This shows that CDP138 are prone to HFD-induced cardiac hypertrophy. Additionally, Masson's trichrome staining of myocardial section from mid-left ventricular wall revealed that hearts from female CDP138 knockout mice KO fed with HFD had more fibrosis as indicated with collagen blue staining than those from and WT HFD mice (FIG. 2, Lower panel). This data shows that CDP138 is important for protecting heart from HFD-induced pathological changes including hypertrophy and fibrosis. Therefore, upregulation of CDP138 can have a beneficial effect on heart function.

The above data shows that CDP138 can be a new drug target for treatment of obesity, diabetes and myocardial dysfunctions.

### REFERENCES

Alessi, D. R., Caudwell, F. B., Andjelkovic, M., Hemmings, B. A., and Cohen, P. (1996). Molecular basis for the substrate specificity of protein kinase B; comparison with MAPKAP kinase-1 and p70 S6 kinase. FEBS Lett 399, 333-338.
Alessi, D. R., James, S. R., Downes, C. P., Holmes, A. B., Gaffney, P. R., Reese, C. B., and Cohen, P. (1997). Characterization of a 3-phosphoinositide-dependent protein kinase which phosphorylates and activates protein kinase Balpha. Curr Biol 7, 261-269.
Bai, J., and Chapman, E. R. (2004). The C2 domains of synaptotagmin—partners in exocytosis. Trends Biochem Sci 29, 143-151.
Bai, L., Wang, Y., Fan, J., Chen, Y., Ji, W., Qu, A., Xu, P., James, D. E., and Xu, T. (2007). Dissecting multiple steps of GLUT4 trafficking and identifying the sites of insulin action. Cell Metab 5, 47-57.
Bryant, N. J., Govers, R., and James, D. E. (2002). Regulated transport of the glucose transporter GLUT4. Nat Rev Mol Cell Biol 3, 267-277.
Calera, M. R., Martinez, C., Liu, H., Jack, A. K., Birnbaum, M. J., and Pilch, P. F. (1998). Insulin increases the association of Akt-2 with Glut4-containing vesicles. J Biol Chem 273, 7201-7204.
Cantley, L. C. (2002). The phosphoinositide 3-kinase pathway. Science 296, 1655-1657.
Chen, X., Al-Hasani, H., Olausson, T., Wenthzel, A. M., Smith, U., and Cushman, S. W. (2003). Activity, phosphorylation state and subcellular distribution of GLUT4-targeted Akt2 in rat adipose cells. J Cell Sci 116, 3511-3518.
Chen, X. W., Leto, D., Chiang, S. H., Wang, Q., and Saltiel, A. R. (2007). Activation of RalA is required for insulin-stimulated Glut4 trafficking to the plasma membrane via the exocyst and the motor protein Myolc. Dev Cell 13, 391-404.
Cho, H., Mu, J., Kim, J. K., Thorvaldsen, J. L., Chu, Q., Crenshaw, E. B., 3rd, Kaestner, K. H., Bartolomei, M. S., Shulman, G. I., and Birnbaum, M. J. (2001). Insulin resistance and a diabetes mellitus-like syndrome in mice lacking the protein kinase Akt2 (PKB beta). Science 292, 1728-1731.
Eguez, L., Lee, A., Chavez, J. A., Miinea, C. P., Kane, S., Lienhard, G. E., and McGraw, T. E. (2005). Full intracellular retention of GLUT4 requires AS160 Rab GTPase activating protein. Cell Metab 2, 263-272.
George, S., Rochford, J. J., Wolfrum, C., Gray, S. L., Schinner, S., Wilson, J. C., Soos, M. A., Murgatroyd, P. R., Williams, R. M., Acerini, C. L., et al. (2004). A family with severe insulin resistance and diabetes due to a mutation in AKT2. Science 304, 1325-1328.
Gonzalez, E., and McGraw, T. E. (2006). Insulin signaling diverges into Akt-dependent and -independent signals to regulate the recruitment/docking and the fusion of GLUT4 vesicles to the plasma membrane. Mol Biol Cell 17, 4484-4493.
Hill, M. M., Clark, S. F., Tucker, D. F., Birnbaum, M. J., James, D. E., and Macaulay, S. L. (1999). A role for protein kinase Bbeta/Akt2 in insulin-stimulated GLUT4 translocation in adipocytes. Mol Cell Biol 19, 7771-7781.
Huang, S., and Czech, M. P. (2007). The GLUT4 glucose transporter. Cell Metab 5, 237-252.
Jiang, L., Fan, J., Bai, L., Wang, Y., Chen, Y., Yang, L., Chen, L., and Xu, T. (2008). Direct quantification of fusion rate reveals a distal role for AS160 in insulin-stimulated fusion of GLUT4 storage vesicles. J Biol Chem 283, 8508-8516.
Jiang, Z. Y., Chawla, A., Bose, A., Way, M., and Czech, M. P. (2002). A phosphatidylinositol 3-kinase-independent insulin signaling pathway to N-WASP/Arp2/3/F-actin required for GLUT4 glucose transporter recycling. J Biol Chem 277, 509-515.
Jiang, Z. Y., Zhou, Q. L., Coleman, K. A., Chouinard, M., Boese, Q., and Czech, M. P. (2003). Insulin signaling through Akt/protein kinase B analyzed by small interfering RNA-mediated gene silencing. Proc Natl Acad Sci USA 100, 7569-7574.
Kandror, K. V., and Pilch, P. F. (2006). Isolation of GLUT4 STorage Vesicles (John Wiley & Sons, Inc.). Curr Protoc Cell Biol, 3.20.1-3.20.13
Kasuga, M., Karlsson, F. A., and Kahn, C. R. (1982). Insulin stimulates the phosphorylation of the 95,000-dalton subunit of its own receptor. Science 215, 185-187.
Kohn, A. D., Summers, S. A., Birnbaum, M. J., and Roth, R. A. (1996). Expression of a constitutively active Akt Ser/Thr kinase in 3T3-L1 adipocytes stimulates glucose uptake and glucose transporter 4 translocation. J Biol Chem 271, 31372-31378.
Koumanov, F., Jin, B., Yang, J., and Holman, G. D. (2005). Insulin signaling meets vesicle traffic of GLUT4 at a plasma-membrane-activated fusion step. Cell Metab 2, 179-189.
Kruger, M., Kratchmarova, I., Blagoev, B., Tseng, Y. H., Kahn, C. R., and Mann, M. (2008). Dissection of the insulin signaling pathway via quantitative phosphoproteomics. Proc Natl Acad Sci USA 105, 2451-2456.
Kupriyanova, T. A., and Kandror, K. V. (1999). Akt-2 binds to Glut4-containing vesicles and phosphorylates their component proteins in response to insulin. J Biol Chem 274, 1458-1464.
Lopez, J. A., Burchfield, J. G., Blair, D. H., Mele, K., Ng, Y., Vallotton, P., James, D. E., and Hughes, W. E. (2009). Identification of a distal GLUT4 trafficking event controlled by actin polymerization. Mol Biol Cell 20, 3918-3929.
Martin, S. S., Haruta, T., Morris, A. J., Klippel, A., Williams, L. T., and Olefsky, J. M. (1996). Activated phosphatidylinositol 3-kinase is sufficient to mediate actin rearrangement and GLUT4 translocation in 3T3-L1 adipocytes. J Biol Chem 271, 17605-17608.
Ng, Y., Ramm, G., Lopez, J. A., and James, D. E. (2008). Rapid activation of Akt2 is sufficient to stimulate GLUT4 translocation in 3T3-L1 adipocytes. Cell Metab 7, 348-356.
Obata, T., Yaffe, M. B., Leparc, G. G., Piro, E. T., Maegawa, H., Kashiwagi, A., Kikkawa, R., and Cantley, L. C. (2000). Peptide and protein library screening defines optimal substrate motifs for AKT/PKB. J Biol Chem 275, 36108-36115.
Okada, T., Kawano, Y., Sakakibara, T., Hazeki, O., and Ui, M. (1994). Essential role of phosphatidylinositol 3-kinase in insulin-induced glucose transport and antilipolysis in rat adipocytes. Studies with a selective inhibitor wortmannin. J Biol Chem 269, 3568-3573.
Olsen, J. V., Blagoev, B., Gnad, F., Macek, B., Kumar, C., Mortensen, P., and Mann, M. (2006). Global, in vivo, and site-specific phosphorylation dynamics in signaling networks. Cell 127, 635-648.
Qin, S., Pande, A. H., Nemec, K. N., and Tatulian, S. A. (2004). The N-terminal alpha-helix of pancreatic phospholipase A2 determines productive-mode orientation of the enzyme at the membrane surface. J Mol Biol 344, 71-89.
Saltiel, A. R., and Kahn, C. R. (2001). Insulin signalling and the regulation of glucose and lipid metabolism. Nature 414, 799-806.
Sano, H., Kane, S., Sano, E., Miinea, C. P., Asara, J. M., Lane, W. S., Garner, C. W., and Lienhard, G. E. (2003). Insulin-stimulated phosphorylation of a Rab GTPase-activating protein regulates GLUT4 translocation. J Biol Chem 278, 14599-14602.
Sarbassov, D. D., Guertin, D. A., Ali, S. M., and Sabatini, D. M. (2005). Phosphorylation and regulation of Akt/PKB by the rictor-mTOR complex. Science 307, 1098-1101.
Sun, X. J., Rothenberg, P., Kahn, C. R., Backer, J. M., Araki, E., Wilden, P. A., Cahill, D. A., Goldstein, B. J., and White, M. F. (1991). Structure of the insulin receptor substrate IRS-1 defines a unique signal transduction protein. Nature 352, 73-77.
Tengholm, A., and Meyer, T. (2002). A PI3-kinase signaling code for insulin-triggered insertion of glucose transporters into the plasma membrane. Curr Biol 12, 1871-1876.
Thurmond, D. C., and Pessin, J. E. (2001). Molecular machinery involved in the insulin-regulated fusion of GLUT4-containing vesicles with the plasma membrane (review). Mol Membr Biol 18, 237-245.
van Dam, E. M., Govers, R., and James, D. E. (2005). Akt activation is required at a late stage of insulin-induced GLUT4 translocation to the plasma membrane. Mol Endocrinol 19, 1067-1077.
Wang, Q., Somwar, R., Bilan, P. J., Liu, Z., Jin, J., Woodgett, J. R., and Klip, A. (1999). Protein kinase B/Akt participates in GLUT4 translocation by insulin in L6 myoblasts. Mol Cell Biol 19, 4008-4018.
Zaid, H., Antonescu, C. N., Randhawa, V. K., and Klip, A. (2008). Insulin action on glucose transporters through molecular switches, tracks and tethers. Biochem. J 413, 201-215.
Zhou, Q. L., Jiang, Z. Y., Mabardy, A. S., Del Campo, C. M., Lambright, D. G., Holik, J., Fogarty, K. E., Straubhaar, J., Nicoloro, S., Chawla, A., et al. (2010). A novel pleckstrin homology domain-containing protein enhances insulin-stimulated Akt phosphorylation and GLUT4 translocation in adipocytes. J Biol Chem 285, 27581-27589.
Bers D M, Patton C W, Nuccitelli R. (1994) A practical guide to the preparation of Ca2+ buffers. Methods Cell Biol. 40:3-29.
Chen, X., Al-Hasani, H., Olausson, T., Wenthzel, A. M., Smith, U., and Cushman, S. W. (2003). Activity, phosphorylation state and subcellular distribution of GLUT4-targeted Akt2 in rat adipose cells. J Cell Sci 116, 3511-3518.
Cox, J., Matic, I., Hilger, M., Nagaraj, N., Selbach, M., Olsen, J. V., Mann M. A. (2009) practical guide to the MaxQuant computational platform for SILAC-based quantitative proteomics. Nat Protoc 4(5):698-705.
Jiang, L., Fan, J., Bai, L., Wang, Y., Chen, Y., Yang, L., Chen, L., and Xu, T. (2008). Direct quantification of fusion rate reveals a distal role for AS160 in insulin-stimulated fusion of GLUT4 storage vesicles. J Biol Chem 283, 8508-8516.
Jiang, Z. Y., Zhou, Q. L., Coleman, K. A., Chouinard, M., Boese, Q., and Czech, M. P. (2003). Insulin signaling through Akt/protein kinase B analyzed by small interfering RNA-mediated gene silencing. Proc Natl Acad Sci USA 100, 7569-7574.
Kandror, K. V., and Pilch, P. E. (2006). Isolation of GLUT4 storage vesicles. (John Wiley & Sons, Inc.). Curr Protoc in Cell Biol 3.20.1-3.20.13
Olsen, J. V., Macek, B. (2009) High accuracy mass spectrometry in large-scale analysis of protein phosphorylation. Methods Mol Biol 492:131-42.
Qin, S., Pande, A. H., Nemec, K. N., and Tatulian, S. A. (2004). The N-terminal alpha-helix of pancreatic phospholipase A2 determines productive-mode orientation of the enzyme at the membrane surface. J Mol Biol 344, 71-89.
Rappsilber, J., Mann, M., Ishihama, Y. (2007). Protocol for micro-purification, enrichment, prefractionation and storage of peptides for proteomics using StageTips. Nat Protoc 2:1896-906.
Shevchenko, A., Tomas, H., Havli, J., Olsen, J. V., & Mann, M. (2007). In-gel digestion for mass spectrometric characterization of proteins and proteomes. Nat Protoc 1, 2856-2860

Throughout this application various publications have been referenced. The disclosures of these publications in their entireties, including GenBank and GI number publications, are hereby incorporated by reference in this application in order to more fully describe the state of the art to which this invention pertains. Although the invention has been described with reference to the examples provided above, it should be understood that various modifications can be made without departing from the spirit of the invention.

1. A method for identifying an agent that modulates 138-kDa C2 domain-containing phosphoprotein (CDP138) activity comprising (a) contacting a cell with a candidate agent, wherein said cell expresses a CDP138 polypeptide or active fragment thereof and (b) detecting CDP138 activity, wherein increased or decreased CDP138 activity in said cell compared to a control cell indicates that said candidate agent is an agent that modules CDP138 activity.

2. The method of claim 1, wherein contacting said cell occurs in vitro.

3. The method of claim 1, wherein said CDP138 activity is binding Ca²⁺ or lipid membranes or inducing fusion of GLUT4 vesicles with a plasma membrane.

4. (canceled)

5. The method of claim 1, wherein said CDP138 activity is increased or decreased.

6. (canceled)

7. The method of claim 1, wherein said cells are further contacted with insulin or a functional equivalent thereof.

8-10. (canceled)

11. The method of claim 1, wherein said CDP138 polypeptide comprises a mutation or a modification at a position selected from the group consisting of S197, S260, S262, S295, T314, T330, S597, S652, S726, S855, D19, D26, D76, D78, D84 and the C2 domain of CDP138.

12. (canceled)

13. A method for identifying an agent that alters phosphorylation of 138-kDa C2 domain-containing phosphoprotein (CDP138) comprising (a) contacting CDP138 or a fragment thereof with a candidate agent under conditions that allow phosphorylation of said CDP138 or fragment thereof and (b) detecting the phosphorylation level of said CDP138 or fragment thereof, wherein altered phosphorylation levels of said CDP138 or fragment thereof indicates that said candidate agent effectively alters the phosphorylation of CDP138.

14. The method of claim 13, wherein contacting said CDP138 occurs in vitro.

15. (canceled)

16. The method of claim 13, wherein said altered phosphorylation of CDP138 is increased phosphorylation or decreased phosphorylation.

17-19. (canceled)

20. The method of claim 13, wherein said CDP138 comprises a mutation or a modification at a position selected from the group consisting of S197, S260, S262, S295, T314, T330, S597, S652, S726, S855, D19, D26, D76, D78, D84 and the C2 domain of CDP138.

21. The method of claim 13, further comprising contacting said CDP138 or fragment thereof with a kinase selected from the group consisting of Akt1, Akt2, CDK, mTOR and CaMKII to phosphorylate CDP138.

22. A method to prolong survival of a neuronal cell comprising introducing into said neuronal cell a nucleic acid molecule encoding 138-kDa C2 domain-containing phosphoprotein (CDP138) or an active fragment thereof, whereby expression of said CDP138 or an active fragment thereof prolongs the survival of said cell.

23. The method of claim 22, wherein said survival occurs in vitro.

24. The method of claim 22, wherein said neuronal cell is a human neuronal cell.

25-27. (canceled)

28. A method for ameliorating or preventing a condition associated with release of insulin from insulin producing cells and insulin-stimulated glucose metabolism in an individual comprising administering an effective amount of an agent that modulates 138-kDa C2 domain-containing phosphoprotein (CDP138) activity in said individual afflicted with said condition, whereby said condition is ameliorated or prevented.

29. (canceled)

30. The method of claim 28, wherein said condition is selected from the group consisting of diabetes mellitus type 1, diabetes mellitus type 2 and a neurodegenerative disease.

31. (canceled)

32. The method of claim 28, wherein said modulation of CDP138 activity comprises binding Ca²⁺, binding lipid membranes or inducing fusion of GLUT4 vesicles with a plasma membrane.

33. (canceled)

34. A method for inhibiting proliferation of a cancer cell comprising contacting said cancer cell with an agent that modulates 138-kDa C2 domain-containing phosphoprotein (CDP138) activity in said cancer cell, whereby increased activity of said CDP138 inhibits said cancer cell, thereby inhibiting proliferation of said cancer cell.

35-37. (canceled)

38. A method for inducing cell cycle arrest of a cancer cell comprising introducing into said cancer cell a nucleic acid molecule encoding 138-kDa C2 domain-containing phosphoprotein (CDP138) or an active fragment or mutant thereof, whereby expression of said CDP138 or active fragment or mutant thereof induces cell cycle arrest of said cancer cell.

39-42. (canceled)

43. A transgenic mouse whose genome comprises a null allele of the gene encoding 138-kDa C2 domain-containing phosphoprotein (CDP138), wherein said mouse exhibits high-fat diet-induced insulin resistance, glucose intolerance, heart hypertrophy or fibrosis.

44-45. (canceled)

46. A method for identifying an agent that modulates insulin resistance, glucose intolerance, heart hypertrophy or fibrosis formation comprising administering a candidate agent to a transgenic mouse of claim 43, determining a level of insulin resistance, glucose intolerance, heart hypertrophy or fibrosis formation in said transgenic mouse administered with said agent, comparing said level to a control level of insulin resistance, glucose intolerance, heart hypertrophy or fibrosis formation, and wherein altered levels of insulin resistance, glucose intolerance, heart hypertrophy or fibrosis formation indicates that said candidate agent modulates insulin resistance, glucose intolerance, heart hypertrophy or fibrosis formation.

47. (canceled)
